(19) United States
(12) Patent Application Publication
(10) Pub. No.: US 2016/0024600 A1
(43) Pub. Date: Jan. 28, 2016

(54) COINCIDENCE REPORTER GENE SYSTEM

(71) Applicant: THE UNITED STATES OF AMERICA, AS REPRESENTED BY THE SECRETARY, DEPARTMENT OF HEALTH AND HUMAN SER, Bethesda, MD (US)

(72) Inventors: James Inglese, Bethesda, MD (US); Ken Chih-Chien Cheng, Rockville, MD (US); Samuel Hasson, Portland, OR (US)

(21) Appl. No.: 14/775,293

(22) PCT Filed: Mar. 15, 2013
(86) PCT No.: PCT/US2013/032184

§ 371 Date: Sep. 11, 2015

(51) Int. Cl. C12Q 1/68

(52) U.S. Cl. CPC C12Q 1/6897

(57) ABSTRACT

Disclosed is a nucleic acid comprising a nucleotide sequence encoding (i) two or more reporters comprising a first reporter and a second reporter that is different from the first reporter; and (ii) one or more ribosomal skip sequences, wherein a ribosomal skip sequence is positioned between the first and second reporters, wherein the first and second reporters are stoichiometrically co-expressed from the nucleotide sequence and the nucleic acid does not comprise a cytomegalovirus-immediate early (CMV-IE) promoter. Also disclosed are methods of screening test compounds for ability to modulate a biological activity of interest using the nucleic acid, as well as related recombinant expression vectors, host cells, and populations of cells.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 167,451 Byte ASCII (Text) file named “712190_ST25.txt,” dated Mar. 14, 2013.

### BACKGROUND OF THE INVENTION

Nucleotide sequences encoding reporters may be useful for any of a variety of applications such as, for example, cell-based assays which may, in turn, be useful for any of a variety of applications including, for example, screening chemical libraries. However, several obstacles to the successful use of reporters in cell-based assays exist. For example, a library compound being screened may interact with the reporter itself instead of the intended biological target, providing misleading results, which may be of a counterintuitive nature. Differences in the conditions of conventional assays can also affect the sensitivity of a given reporter, which may also provide misleading data. Such occurrences may cause compounds of interest to be overlooked and/or may make it necessary for investigators to dedicate considerable additional time and effort to sort through the results to eliminate the false positive results and/or false negative results.

Accordingly, there exists a need for improved nucleotide sequences encoding reporters and cell-based assays.

### BRIEF SUMMARY OF THE INVENTION

The invention provides a method of screening test compounds for ability to modulate a biological activity of interest, the method comprising: (a) introducing a nucleic acid into a population of cells, wherein (i) the nucleic acid comprises a nucleotide sequence encoding two or more reporters including a first reporter and a second reporter that is different from the first reporter, (ii) the nucleic acid further comprises a nucleotide sequence encoding one or more ribosomal skip sequences, wherein a ribosomal skip sequence is positioned between nucleotide sequences encoding the first and second reporters, and (iii) the first and second reporters are stoichiometrically co-expressed under control of a transcriptional regulatory element (TRE) and/or promoter that is activated or repressed by modulation of the biological activity of interest; (b) dividing the cells of (a) into more than one sub-population; (c) culturing each sub-population of cells with a test compound, wherein each sub-population is cultured with a different test compound; (d) measuring expression of the first and second reporters in each cultured sub-population of cells; and (e) identifying at least one test compound modulating the biological activity of interest when both of the first and second reporters are expressed by the sub-population of cells that was cultured with the test compound or when a basal level of expression of both of the first and second reporters is repressed or increased in the sub-population of cells that is cultured with the test compound.

Another embodiment of the invention provides a method of diagnosing a subject as having a condition, the method comprising: (a) obtaining a sample from the subject, wherein the sample is suspected of containing an analyte associated with the condition; (b) introducing a nucleic acid into a population of cells, wherein (i) the nucleic acid comprises a nucleotide sequence encoding two or more reporters comprising a first reporter and a second reporter that is different from the first reporter, and (ii) the first and second reporters are stoichiometrically co-expressed under control of a transcriptional regulatory element and/or promoter that is activated or repressed in the presence of the analyte; (c) culturing the cells with the sample suspected of containing the analyte; (d) measuring expression of the first and second reporters by the cultured cells; and (e) diagnosing the patient as having the condition when both of the first and second reporters are expressed by the cultured cells.

Still another embodiment of the invention provides a kit for screening test compounds for ability to modulate a biological activity of interest, the kit comprising: (a) (i) a nucleic acid comprising a nucleotide sequence encoding two or more reporters including a first reporter and a second reporter that is different from the first reporter and one or more ribosomal skip sequences, wherein a ribosomal skip sequence is positioned between the first and second reporters, wherein the first and second reporters are stoichiometrically co-expressed from the nucleotide sequence, and/or (ii) a population of cells comprising the nucleic acid; and (b) at least one container for holding the nucleic acid or population of cells.

Still another embodiment of the invention provides a kit for diagnosing a subject as having a condition, the kit comprising: (a) (i) a nucleic acid comprising a nucleotide sequence encoding two or more reporters including a first reporter and a second reporter that is different from the first reporter and one or more ribosomal skip sequences, wherein a ribosomal skip sequence is positioned between the first and second reporters, wherein the first and second reporters are stoichiometrically co-expressed from the nucleotide sequence, and/or (ii) a population of cells comprising the nucleic acid; and (b) at least one container for holding the nucleic acid or population of cells.

Additional embodiments of the invention provide related nucleic acids, recombinant expression vectors, host cells, and populations of cells.

### BRIEF DESCRIPTION OF THE SEVERAL VIEWS OF THE DRAWING(S)

FIGS. 1A and 1B are graphs showing the bioluminescent output for FLuc (A) or RLuc (B) as measured by Relative Luminescent Units (RLU) for non-transfection control (transfection reagent only) (lane 1); SV40-driven FLuc mono-reporter (pGL3-Control) (lane 2); and FLuc-P2A-RLuc dual reporter (pCI-6.20) (lane 3). Data plotted are average of replicate (n=2) determinations; error bars represent standard deviation (s.d.).

FIGS. 2A-2B are graphs showing the bioluminescent output for FLuc (A) or RLuc (B) as measured by RLU for cells transfected with the cAMP-response element (CRE)-driven pCl-6.24 construct in response to treatment with Forskolin (), the FLuc ligand PCT124 (▴) that stabilizes the reporter enzyme at low concentration but inhibits at high concentrations, or the RLuc ligand BTS (▾) over a concentration range from 0.01 nM to 100 μM. Data plotted are average of replicate (n=2) determinations; error bars represent standard deviation (s.d.).

FIG. 3 is a graph showing the EC₅₀ correlation plot for compounds activating FLuc and RLuc expression equally; r²=0.87. Three classes of compounds were identified, purinergic Y2 receptor agonists, (closed circles), a muscarinic receptor agonist, compound 18 (open circle), and the adenylyl cyclase activator forskolin (FSK) (square). EC₅₀ of compounds that selectively increased RLuc (triangles) are plotted along the x-axis. Data plotted are average of replicate (n=2) determinations; error bars represent standard deviation (s.d.).

FIGS. 4A-4D and 4I-4K are graphs showing reporter gene activation concentration response curves (percent activity) as measured in a cell-based quantitative high throughput screen (qHTS) using the FLuc (solid squares) and RLuc (solid circles) reporters with RLuc cell-based activator compound (cpd) 20 (A), cpd 21 (B), cpd 22 (C), cpd 23 (D), cpd 24 (I), cpd 25 (J), or cpd 26 (K). Data plotted are average of replicate (n=2) determinations; error bars represent standard deviation (s.d.).

FIGS. 4E-4H and 4L-4N are graphs showing enzyme inhibition concentration response curves (percent activity) as measured in enzymatic assays using the FLuc (open squares) and RLuc (open circles) reporter enzymes with RLuc cell-based activator compound (cpd) 20 (E), cpd 21 (F), cpd 22 (G), cpd 23 (H), cpd 24 (L), cpd 25 (M), or cpd 26 (N). Data plotted are average of replicate (n=2) determinations; error bars represent standard deviation (s.d.).

FIG. 5A is a graph showing the percent activity measured in the 57 μM concentration level of the qHTS series for the agonists having RLuc (open circle) or FLuc (closed circle) response. Compounds not activating FLuc (x) or RLuc (+) are also shown.

FIG. 5B is a graph showing the percent activity measured in the 57 concentration level of the qHTS series for the agonists having a coincident FLuc (closed circle) and RLuc (open circle) response and, therefore, activating reporter gene transcription via CRE-responsive signaling pathways. Compounds not activating FLuc (x) or RLuc (+) are also shown. Data plotted are average of replicate (n=2) determinations; error bars represent standard deviation (s.d.).

FIGS. 6A and 6B are graphs showing the bioluminescent output for FLuc (A) or the fluorescent output for emGFP (B) as measured by RLU or fluorescence intensity units (FLU), respectively, for cells transfected with a 4XCRE-driven FLuc-P2A-emGFP construct and treated with DMSO or 50 μM forskolin. Data plotted are average of triplicate (n=3) determinations; error bars represent standard deviation (s.d.).

FIGS. 7A and 7B are graphs showing the bioluminescent output for NLucP (A) or the fluorescent output for emGFP (B) as measured by RLU or FLU, respectively, for cells transfected with a 4XCRE-driven NLuc-P2A-emGFP construct and treated with DMSO or 50 forskolin. Data plotted are average of triplicate (n=3) determinations; error bars represent standard deviation (s.d.).

FIGS. 8A and 8B are graphs showing the bioluminescent output for FLuc2P (A) or NLucP (B) as measured by RLU for cells transfected with a p53 RE-driven FLuc2P-P2A-NLucP construct and treated with DMSO or 10 μM etoposide. Data plotted are average of triplicate (n=3) determinations; error bars represent standard deviation (s.d.).

FIGS. 9A and 9B are graphs showing the bioluminescent output for FLuc2P (A) or NLucP (B) as measured by RLU for cells transfected with an ARE-driven FLuc2P-P2A-NLucP constructs and treated with DMSO or 100 μM tBHQ. Data plotted are average of triplicate (n=3) determinations; error bars represent standard deviation (s.d.).

FIGS. 10A-10D are schematics illustrating the genome-editing strategy to generate the Parkin coincidence reporter cell line to report changes in PARK2 (Parkin) gene expression. (A) The PARK2 gene is present in chromosome 6 of the human genome and is composed of a sequence that encodes 12 exons. (B) TALEN-mediated genome editing targeted the first two codons of the PARK2 gene in exon 1, the exon that also contained a 5′ untranslated region (UTR). (C) Replacement of the “ATGATAG” sequence at the 3′ end of exon 1 with the FLuc-P2A-NLuc coincidence reporter cassette followed by a SV40 late poly(A) sequence was accomplished with TALEN-mediated double-strand cleavage of the genomic DNA. This cleavage stimulated homologous recombination in the presence of a donor DNA plasmid containing ˜1 kb of homologous sequence 5′ and 3′ of the coincidence reporter cassette. (D) The final cell line was found to contain the coincidence reporter cassette that had correctly integrated into a single allele of the endogenous PARK2 gene locus.

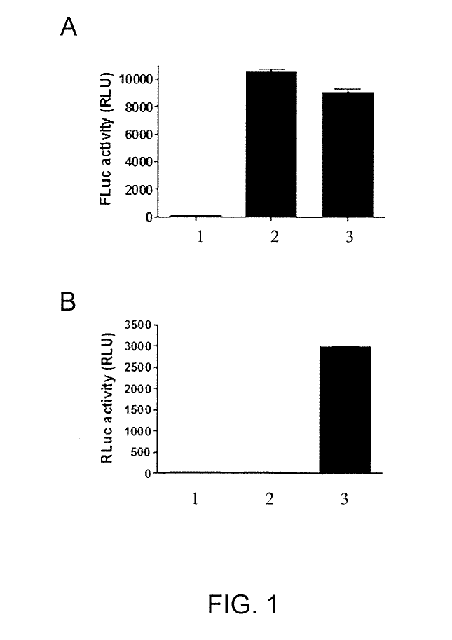

FIG. 1E is a schematic showing the investigation of the regulation of Parkin gene expression. The Parkin coincidence reporter cell line was constructed to investigate the expression of Parkin from the endogenous promoter. Several response elements such as MYC and CREB are known to exist in the Parkin promoter and are regulated by ATF-4, n-MYC, and c-JUN. Higher order regulation has been hypothesized from the JNK pathway and eIF2. However, other response elements may exist that interface with cellular signaling pathways (denoted as “?”). “P” denotes a phosphorylation event.

FIG. 11A is a graph showing the relative parkin mRNA level (normalized to GAPDH) from the Parkin coincidence reporter cell line treated with vehicle only for 24 hours, 10 μM CCCP for 24 hours, or 2 μg/mL Tunicamycin for 12 hours. Data plotted are average of triplicate (n=3) determinations.

FIG. 11B is a graph showing the relative FLuc-P2A-NLuc mRNA level (normalized to actin) from the parkin coincidence reporter parental cell line alone or treated with vehicle only for 24 hours, 10 μM CCCP for 24 hours, or 2 μg/mL Tunicamycin for 12 hours. Data plotted are average of triplicate (n=3) determinations.

FIG. 11C is a graph showing the luminescence signal (RLU) generated by the Parkin coincidence reporter cell line treated with vehicle only (unshaded bars) or a positive control (shaded bars) for R1:FLuc Signal or R2:NLuc Signal. Bars are mean+/−standard deviation of 384 wells per condition.

FIGS. 12A-12E are graphs showing the activity (% of control) of FLuc (squares) or NLuc (circles) upon treating the Parkin coincidence reporter cell line with PTC-124 (A), Resveratrol (B), Nimodipine (C), MG-132 (D), or Quercetin (E).

FIGS. 13A-13B are schematics illustrating nucleotide constructs including a transcriptional response element (TRE) either positively (+) (activating) or negatively (−) (repressing) a promoter (P) driving the expression of the coincidence reporter including a first reporter (R1), a ribosomal skip sequence (RS), and a second reporter (R2), and n is the copy number of R1 and RS (A) or RS and R2 (B) that will be expressed.

### DETAILED DESCRIPTION OF THE INVENTION

It has been discovered that misleading results from cell-based assays may be reduced or avoided by introducing a nucleic acid into a population of cells, wherein (i) the nucleic acid comprises a nucleotide sequence encoding two or more reporters including a first reporter and a second reporter, wherein the second reporter is different from the first reporter, (ii) the nucleic acid further comprises a nucleotide sequence encoding one or more ribosomal skip sequences, wherein a ribosomal skip sequence is positioned between nucleotide sequences encoding the two or more reporters, and (iii) the two or more reporters are stoichiometrically co-expressed under control of a transcriptional regulatory element (TRE) and/or promoter that is activated or repressed by modulation of a biological activity of interest.

When the TRE and/or promoter is activated or repressed by a particular biological activity such as, for example, activation of a cellular receptor by a compound of interest, both reporter genes will be expressed. The probability that a compound of interest will interact with each of two or more different, unrelated reporters instead of the intended biological target is believed to be very low. Therefore, the “coincident” output from both reporters may, advantageously, provide a more reliable measurement of the biological activity under study. For example, the inventive kits, nucleic acids, recombinant expression vectors, host cells, and populations of cells (hereinafter, “cell-based assay materials”) and the inventive methods may, advantageously, make it possible to reduce or avoid misleading results due to the interaction of a compound being screened with the reporter itself instead of the intended biological target and/or differences in assay conditions. Accordingly, the inventive methods and cell-based assay materials may, advantageously, make it possible to reduce or avoid overlooking true compounds of interest and/or spending time and effort sorting through the results to eliminate the false positive results and/or false negative results.

An embodiment of the invention provides a method of screening test compounds comprising: (a) introducing into a population of cells a nucleic acid comprising a nucleotide sequence encoding (i) two or more reporters that are each different from one another and that are all stably stoichiometrically co-expressed under the control of a single transcriptional regulatory element (TRE) and/or promoter, and (ii) a ribosomal skip sequence positioned between each nucleotide sequence encoding a different reporter; and (b) treating the population of cells with one or more test compounds.

An embodiment of the invention provides a method of screening a library of test compounds for ability to modulate a biological activity of interest, the method comprising: (a) introducing a nucleic acid into a population of cells, wherein (i) the nucleic acid comprises a nucleotide sequence encoding two or more reporters including a first reporter and a second reporter that is different from the first reporter, (ii) the nucleic acid further comprises a nucleotide sequence encoding one or more ribosomal skip sequences, wherein a ribosomal skip sequence is positioned between nucleotide sequences encoding the first and second reporters, and (iii) the first and second reporters are stoichiometrically co-expressed under control of a transcriptional regulatory element and/or promoter that is activated or repressed by modulation of the biological activity of interest; (b) dividing the cells of (a) into more than one sub-population; (c) culturing each sub-population of cells with a test compound from the library, wherein each sub-population is cultured with a different test compound from the library; (d) measuring expression of the first and second reporters in each cultured sub-population of cells; and (e) identifying at least one test compound modulating the biological activity of interest when both of the first and second reporters are expressed by the sub-population of cells that was cultured with the test compound or when a basal level of expression of both of the first and second reporters is repressed or increased in the sub-population of cells that is cultured with the test compound.

The method may comprise introducing a nucleic acid into a population of cells, wherein (i) the nucleic acid comprises a nucleotide sequence encoding two or more reporters including a first reporter and a second reporter that is different from the first reporter, (ii) the nucleic acid further comprises a nucleotide sequence encoding one or more ribosomal skip sequences, wherein the ribosomal skip sequence is positioned between nucleotide sequences encoding the first and second reporters, and (iii) the first and second reporters are stoichiometrically co-expressed under control of a transcriptional regulatory element and/or promoter that is activated or repressed by modulation of the biological activity of interest. Introducing a nucleic acid into a population of cells may be carried out in any suitable manner known in the art. See, for example, Green et al. (eds.), Molecular Cloning, A Laboratory Manual, 4^{th} Edition, Cold Spring Harbor Laboratory Press, New York (2012) and Ausubel et al., Current Protocols in Molecular Biology, Greene Publishing Associates and John Wiley & Sons, NY (2007). Introducing the nucleic acid into the population of cells may include, for example, physically contacting the cells with the nucleic acid under conditions that permit uptake of the nucleic acid by the cells such that the cells comprise the nucleic acid and expression of the nucleic acid by the cells. Introducing the nucleic acid into the population of cells may include, for example, transfecting or transducing the cells with the nucleic acid.

The population of cells is not limited and may comprise any type of cell suitable for expressing the nucleic acid and for studying the particular biological activity and/or compounds of interest. The cell can be a eukaryotic cell, e.g., plant, animal, fungi, or algae, or can be a prokaryotic cell, e.g., bacteria or protozoa. The cell can be a cultured cell or a primary cell, i.e., isolated directly from an organism, e.g., a human. The cell can be an adherent cell or a suspended cell, i.e., a cell that grows in suspension. Suitable cells are known in the art and include, for instance, DH5α E. coli cells, Chinese hamster ovarian cells, monkey VERO cells, COS cells, HEK293 cells, and the like. In an embodiment, the cell is a mammalian cell. Preferably, the cell is a human cell. The cell may be any type of mammalian cell including, but not limited to, a T cell, a B cell, a macrophage, a neutrophil, an erythrocyte, a hepatocyte, an endothelial cell, an epithelial cell, a muscle cell, or a brain cell, etc.

The nucleic acid comprises a nucleotide sequence encoding two or more reporters including a first reporter and a second reporter, wherein the second reporter is different from the first reporter. The nucleic acid may comprise a nucleotide sequence encoding any suitable number of different reporters. For example, the nucleic acid may comprise a nucleotide sequence encoding 2, 3, 4, 5, 6, 7, 8, 9, 10, 11, 12, 13, 14, 15, or more reporters.

The reporters may be any reporter known in the art. Suitable reporters may include, but are not limited to, any of fluorescent protein (e.g., green (GFP), red, yellow, or cyan fluorescent protein, enhanced green, red, yellow, or cyan fluorescent protein), beta-lactamase, beta-galactosidase, luciferase (e.g., firefly luciferase (FLuc), Renilla (RLuc) luciferase, NANOLUC luciferase (NlucP) (Promega, Madison, Wis.), bacterial luciferase, Click-Beetle Luciferase Red (CBRluc), Click-Beetle Luciferase Green (CBG68luc and CBG99luc), Metridia pacifica Luciferase (MetLuc), Gaussia Luciferase (GLuc), Cypridina Luciferase, and Gaussia-Dura Luciferase), chloramphenicol acetyltransferase (CAT), neomycin phosphotransferase, alkaline phosphatase, secreted alkaline phosphatase (SEAP), Chloramphenicol acetyltransferase (CAT), mCherry, tdTomato, TurboGFP, TurboRFP, dsRed, dsRed2, dsRed Express, AcGFP1, ZsGreen1, Red Firefly Luciferase, Enhanced Click-Beetle Luciferase (ELuc), Dinoflagellate Luciferase, Pyrophorus plagiophthalamus Luciferase (lucGR), Bacterial luciferase (Lux), pmeLUC, Phrixothrix hirtus Luciferase, Gaussia-Dura Luciferase, RenSP, Vargula hilgendorfii Luciferase, Lucia Luciferase, Metridia longa Luciferase (MetLuc), HaloTag, SNAP-tag, CLIP-tag, β-Glucuronidase, Aequorin, Secreted placental alkaline phosphatase (SPAP), Gemini, TagBFP, mTagBFP2, Azurite, EBFP2, mKalamal, Sirius, Sapphire, T-Sapphire, ECFP, Cerulean, SCFP3A, mTurquoise, mTurquoise2, Midoriishi-Cyan, TagCFP, mTFP1, Emerald, Superfolder GFP, Azami Green, TagGFP2, mUKG, mWasabi, Clover, Citrine, Venus, SYFP2, TagYFP, Kusabira-Orange, mKO, mKO2, mOrange, mOrange2, mRaspberry, mStrawberry, mTangerine, TagRFP, TagRFP-T, mApple, mRuby, mRuby2, mPlum, HcRed-Tandem, mKate2, mNeptune, NirFP, TagRFP657, IFP1.4, iRFP, mKeima Red, LSS-mKate1, LSS-mKate2, PA-GFP, PAmCherry1, PATagRFP, Kaede (green), Kaede (red), KikGR1 (green), KikGR1 (red), PS-CFP2, PS-CFP2, mEos2 (green), mEos2 (red), mEos3.2 (green), mEos3.2 (red), PSmOrange, PSmOrange, Dronpa, TurboYFP, TurboFP602, TurboFP635, TurboFP650, hrGFP, hrGFP II, E2-Crimson, HcRed1, Dendra2, AmCyan1, ZsYellow1, mBanana, EBFP, Topaz, mECFP, CyPet, yPet, PhiYFP, DsRed-Monomer, Kusabira Orange, Kusabira Orange2, Jred, AsRed2, dKeima-Tandem, AQ143, mKikGR, and homologs and variants thereof. The first reporter is different from the second reporter. In an embodiment of the invention, the two or more reporters are different and unrelated so as to reduce or eliminate the probability that a test compound will interfere with the output of two or more (e.g., both) reporters. For example, the two or more reporters may use different substrates and/or mechanisms to produce an output.

The nucleic acid further comprises a nucleotide sequence encoding one or more ribosomal skip sequences, wherein a ribosomal skip sequence is positioned between nucleotide sequences encoding any two or more reporters. The ribosomal skip sequence prevents the formation of a normal peptide bond, resulting in the ribosome skipping to the next codon and releasing the translated polypeptide upstream of the skip sequence. Accordingly, the ribosomal skip sequence provides a single mRNA sequence from which both reporters are translated. The ribosomal skip sequence mediates co-translational cleavage of the two or more reporters at a single cleavage site. The ribosomal skip sequence employed in the inventive methods and cell-based assay materials may be any suitable length. The ribosomal skip sequence may include, for example, from about 15 to about 25 amino acid residues, preferably about 20 amino acid residues. Examples of suitable ribosomal skip sequences include any of SEQ ID NOs: 21-344. In an embodiment, the ribosomal skip sequence is a Picornavirus 2A (P2A) peptide or a homolog or variant thereof. An example of a nucleotide sequence encoding a P2A peptide suitable for use in the inventive methods and cell-based assay materials may comprise a nucleotide sequence comprising SEQ ID NO: 1. An example of a P2A peptide suitable for use in the inventive methods and cell-based assay materials may comprise an amino acid sequence comprising SEQ ID NO: 2.

The nucleic acid may comprise a nucleotide sequence encoding any combination of two or more reporters and a ribosomal skip peptide positioned between different reporters. Examples of the nucleic acids suitable for use in the inventive methods and cell-based assay materials include, but are not limited to, nucleic acids comprising a nucleotide sequence encoding (i) FLuc-P2A-RLuc comprising SEQ ID NO: 3 encoding an amino acid sequence comprising SEQ ID NO: 4; (ii) FLuc-P2A-NLucP comprising SEQ ID NO: 5 encoding an amino acid sequence comprising SEQ ID NO: 6; (iii) FLuc-P2A-GFP comprising SEQ ID NO: 7 encoding an amino acid sequence comprising SEQ ID NO: 8; (iv) NLucP-P2A-GFP comprising SEQ ID NO: 9 encoding an amino acid sequence comprising SEQ ID NO: 10; and (v) NLucP-P2A-beta lactamase comprising SEQ ID NO: 11 encoding an amino acid sequence comprising SEQ ID NO: 12.

The two or more reporters are stoichiometrically co-expressed under control of one or more transcriptional regulatory element (TRE)s and/or promoters that is/are activated or repressed by modulation of a biological activity of interest. In an embodiment of the invention, the nucleic acid comprises no more than a single TRE and/or promoter that induces stable stoichiometric co-expression of all reporters. The TRE and/or promoter may be any suitable TRE and/or promoter known in the art and may be selected on the basis of the particular biological activity under study. For example, to determine whether a test compound activates transcription of a target gene, the two or more reporters may be co-expressed under control of a TRE and/or promoter that controls expression of that target gene. The type and number of copies of the TRE and/or promoter is not limited any may include, for example, any of a positive control element, a negative control element, a steroid response element (e.g., glucocorticoid response element (GRE)), a heat shock response element, a metal response element, a repressor binding site, a hormone response element (e.g., estrogen receptor element (ERE)), a serum response element (SRE), a cAMP-response element (CRE), a 12-O-tetradecanoylphorbol 13-acetate (TPA) response element, 3′,5′-cyclic adenosine monophosphate response element, Abscisic acid (ABA)-response element, Adenosine monophosphate response element, Amino acid response element (AARE), Anaerobic responsive element, Androgen response element, Antioxidant response elements (AREs), aryl hydrocarbon response element, Auxin response element, Bone morphogenetic protein (BMP)-response element, Calcitonin-response element, Calcium-response element, Carbohydrate response element (ChoRE), CD28 response element, Cholesterol response element, CO(2) response element, Copper-responsive elements, Dioxin Response Element, E-box element, Ecdysone response element (EcRE), EGF response element, EGF/TGFalpha response element, Elicitor response element, ER stress response element, EWS/FLI response element, FGF2-response element, G-Box element, Gibberellin-responsive elements, Glucose response element, High-temperature response element, HIV trans-activation response (TAR) element, Human muscle-specific Mt binding site, Hypoxia-response elements (HREs), Insulin responsive element (IRE), Interferon-stimulated response element, Interleukin/cytokine response element, Involucrin promoter transcriptional response element, Iron-responsive element, Jasmonate-responsive element, Lipoprotein Response Element, Low-temperature response element, Lytic switch protein (ORF50) response element, Myc-Max response element, Negative retinoic acid response element, Nerve growth factor-responsive element, Nitrate response element, Nitric oxide response element, Nitrite response element, Nuclear factor 1 response element, Nuclear factor of activated T-cells (NFAT)-response element, Osmotic response element (ORE), p53 response element, PAX-4/PAX-6 paired domain binding sites, P-Box element, Peroxisome proliferator (PP) response element, Peroxisome proliferator-activated receptor alpha response element, Peroxisome proliferator-activated receptor gamma response element, Phorbol ester response element, Plastid response element, Progesterone response element (PRE), Prostaglandin response element, Retinoic acid response element, Retinoid response element, Retinoid X receptor (RXR) binding element, Shear stress response elements (SSREs), Smad Response Element, Sp1 response element, Sugar Response Element, Synaptic activity response element, T-Box element, Tetracycline Response Element (TRE), Thyroid hormone response element, UV response element, UV/blue light-response element, Vitamin D Response Element, VLDL response element (VLDLRE), Wnt/B-catenin/TCF response element, and a Xenobiotic response element. Additional examples of TREs and/or promoters are set forth in Table 1. In an embodiment, the nucleic acid comprises a single promoter sequence that induces stable stoichiometric co-expression of all of the reporters. The TRE and/or promoter may be viral, eukaryotic, or prokaryotic in origin. In an embodiment, the TRE comprises p53 (SEQ ID NO: 367), ARE (SEQ ID NO: 368), or a CRE nucleotide sequence comprising SEQ ID NO: 13 (CRE) or SEQ ID NO: 14 (4XCRE).

TABLE 1

| Family | Full Name | Members (Official Gene Symbols) |
|---|---|---|
| AP1 | Activator Protein 1 | FOS, FOSB, JUN, JUNB, JUND |
| AP2 | Activator Protein 2 | TFAP2A, TFAP2B, TFAP2C, TFAP2D, TFAP2E |
| AR | Androgen Receptor | AR |
| ATF | Activating Transcription Factor | ATF1 - 7 |
| BCL | B-cell CLL/lymphoma | BCL3, BCL6 |
| BRCA | breast cancer susceptibility protein | BRCA1 - 3 |
| CEBP | CCAAT/enhancer binding protein | CEBPA, CEBPB, CEBPD, CEBPE, CEBPG |
| CREB | cAMP responsive element binding protein | CREB1 - 5, CREM |
| E2F | E2F transcription factor | E2F1 - 7 |
| EGR | early growth response protein | EGR1 - 4 |
| ELK | member of ETS oncogene family | ELK1, ELK3, ELK4 |
| ER | Estrogen Receptor | ESR1, ESR2 |
| ERG | ets-related gene | ERG |
| ETS | ETS-domain transcription factor | ETS1, ETS2, ETV4, SPI1 |
| FLI1 | friend leukemia integration site1 | FLI1 |
| GLI | glioma-associated oncogene homolog | GLI1 - 4 |
| HIF | Hypoxia-inducible factor | HEF1A, ARNT, EPAS1, HIF3A |
| HLF | hepatic leukemia factor | HLF |
| HOX | homeobox gene | HOXA, HOXB, HOXD series, CHX10, MSX1, MSX2, TLX1, PBX2 |
| LEF | lymphoid enhancing factor | LEF1 |
| MYB | myeloblastosis oncogene | MYB, MYBL1, MYBL2 |
| MYC | myelocytomatosis viral oncogene homolog | MYC |
| NFI | nuclear factor I; CCAAT-binding transcription factor | NFIA, NFIB, NFIC, NFIX |
| NFKB | Nuclear factor kappa B, reticuloendotheliosis oncogene | NFKB1, NFKB2, RELA, RELB, REL |
| OCT | Octamer binding proteins | POU2F1 - 3, POU3F1 - 2, POU5F1 |
| p53 | P53 family | TP53, TP73L, TP73 |
| PAX | paired box gene | PAX1 - 9 |
| PPAR | Peroxisome proliferator-activated receptor | PPARA, PPARD, PPARG |
| PR | Progesterone Receptor | PGR |
| RAR | retinoic acid receptor | RARA, RARB, RARG |
| SMAD | Mothers Against Decapentaplegic homolog | SMAD1 - 9 |
| SP | sequence-specific transcription factor | SP1 - 8 |
| STAT | signal transducer and activator of transcription | STAT1 - 6 |
| TAL1 | T-cell acute lymphocytic leukemia-1 protein | TAL1 |
| USF | upstream stimulatory factor | USF1, USF2 |
| WT1 | Wilms tumor 1 (zinc finger protein) | WT1 |

The transcription of the two or more reporters is under control of the same TRE and/or promoter such that the two or more reporters are stoichiometrically co-expressed. “Stoichiometrically co-expressed,” as used herein, refers to the co-expression of two or more reporters in a stable, non-varying ratio that is proportional to the number of copies of each reporter encoded by the inventive nucleic acids. The inventive nucleic acid may include any number of copies of any given reporter.

In an embodiment, the nucleic acid further comprises one or more nucleotide sequences that may be useful for directing the integration of the nucleic acid into a specific target site in the genome of the population of cells. In this regard, the nucleic acid may further comprise nucleotide sequences flanking a combination of the nucleotide sequences encoding the two or more reporters and the one or more ribosomal skip sequences and, optionally, the TRE and/or promoter, wherein the flanking nucleotide sequences are homologous to a left and right arm of a target site in a genome of the population of cells. In an embodiment of the invention, the nucleic acid may further comprise nucleotide sequences flanking a combination of the nucleotide sequences encoding the two or more reporters and the one or more ribosomal skip sequences without a TRE and/or promoter, wherein the flanking nucleotide sequences are homologous to a left and right arm of a target site in a genome of the population of cells, such that the nucleic acid may be integrated into a genome target site such that expression of the reporters is under the control of a TRE and/or promoter of interest that is endogenous to the population of cells. The nucleotide sequences homologous to left and right aims of the genome target site may be any suitable size that provides for insertion of the nucleic acid in the target site.

The biological activity of interest may be any biological activity that is modulated by one or more test compounds being screened. Modulation may include any change in the biological activity that occurs in the presence of the test compound as compared to in the absence of the test compound. Modulation may include, for example, stimulation or repression of a biological activity of interest. Suitable biological activities may include, but are not limited to, any one or more of modulation of expression of a target gene, activation or repression of a cellular receptor, transcriptional and epigenetic processes, host cell-pathogen interactions, cell differentiation, metabolic adaptation, stress-induced response, cell division, cell death, cell senescence, cell-fate reprogramming, pluripotency induction, metastasis, oncogenic transformation, cell morphology alteration, inflammatory response, cellular migration, extracellular matrix/substrate interaction, autophagic stimulation, ubiquitin-proteasome response, genetic repair induction, organellar biogenesis, unfolded-protein response, electrochemical signaling, neurotransmitter response, and general activation or repression of intracellular or extracellular cell signaling pathways. The biological activity is not limited and may include any biological activity. For example, the biological activity may be adenylyl cyclase signaling through the cAMP-response element (CRE) or transcription from the PARK2 gene promoter.

The method may comprise dividing the cells comprising the nucleic acid into more than one sub-population. In an embodiment, the cells are divided into at least two sub-populations. Dividing the cells comprising the nucleic acid into more than one sub-population may be carried out in any suitable manner. For example, the cells may be divided by being placed in different wells of multi-well plates.

The method may comprise culturing (e.g., treating) each sub-population of cells with a test compound from a library, wherein each sub-population is cultured with a different test compound from the library. The library may comprise any collection of two or more test compounds that is believed to possibly contain one or more compounds that may modulate the biological activity of interest. Each sub-population of cells is cultured with a different test compound such that the ability of each compound to modulate the biological activity of interest may be evaluated.

The method may comprise measuring expression of the two or more reporters in each cultured sub-population of cells. Modulation of the biological activity of interest by one or more test compounds directly or indirectly activates or represses the TRE and/or promoter which, in turn, activates or represses expression of the two or more reporters. Measuring expression of the two or more reporters may be carried out in any suitable manner. For example, measuring expression of the two or more reporters may include contacting the cultured cells with one or more detection reagents that react(s) with the first and/or second reporters to provide a detectable indicator (e.g., fluorescence, luminescence, and color changes) of the presence or absence of the first and/or second reporter, respectively. The detectable indicator may, for example, be a visible indicator. Measuring expression of the two or more reporters may include observing and/or measuring the quantity of any one or more of fluorescence, luminescence, absorbance, and color changes, as is appropriate for particular reporters chosen. In an embodiment of the invention in which the reporters chosen do not require a detection reagent in order to provide a detectable indicator of the presence or absence of the reporter (e.g., any of the fluorescent proteins such as green, red, yellow, or cyan fluorescent protein), measuring expression of the two or more reporters may be carried out without contacting the cultured cells with a detection reagent. In an embodiment of the invention in which the first reporter chosen does not require a detection reagent in order to provide a detectable indicator of the presence or absence of the reporter and the second reporter chosen requires a detection reagent, measuring expression of the first reporter may be carried out without contacting the cultured cells with a detection reagent and measuring the expression of the second reporter may be carried out by contacting the cultured cells with a detection reagent.

In an embodiment of the invention in which the two or more reporters chosen both require a detection reagent in order to provide a detectable indicator of the presence or absence of the reporters, measuring expression of the first reporter may be carried out by contacting the cultured cells with a first detection reagent and measuring the expression of the second reporter may be carried out by contacting the cultured cells with a second detection reagent. When two or more detection reagents are used, the method may comprise contacting the cultured cells with the first and second detection reagents sequentially. In this regard, the method may comprise first contacting the cultured cells with a first detection reagent to provide a first detectable indicator and secondly contacting the cultured cells with a second detection reagent to provide a second detectable indicator. In an embodiment of the invention, the method comprises measuring the level of activity or expression of the reporters in the cells.

The method may comprise identifying at least one test compound modulating the biological activity of interest when all of the two or more reporters (e.g., both of the first and second reporters) are expressed by the sub-population of cells that was cultured with the test compound. If none of the two or more reporters (e.g., none of the first and second reporters) are expressed upon culture with a given test compound, then that test compound may be identified as not stimulating or repressing the biological activity of interest. If less than all of the reporters, e.g., only one of the two or more reporters (e.g., only one of the first and second reporters) are expressed upon culture with a given test compound, then that test compound may be identified as not stimulating or repressing the biological activity of interest and, instead, may be identified as interfering with the expression of one of the reporters. If all of the two or more reporters (e.g., both the first and second reporters) are expressed upon culture with a given test compound, then that test compound may be identified as stimulating or repressing the biological activity of interest. The probability that a compound of interest will interact with two or more reporters (e.g., both of the first and second reporters) instead of stimulating or repressing the biological activity of interest is believed to be very low. Accordingly, the inventive methods and cell-based assay materials are believed to provide a more reliable measure of the ability of a given test compound to modulate the biological activity of interest.

The method may comprise identifying at least one test compound modulating the biological activity of interest when the expression of all of the two or more reporters (e.g., both of the first and second reporters) is repressed or increased from a basal level in the sub-population of cells that was cultured with the test compound. If the expression of none of the two or more reporters (e.g., none of the first and second reporters) is repressed or increased from a basal level upon culture with a given test compound, then that test compound may be identified as not stimulating or repressing the biological activity of interest. If the expression of less than all of the reporters, e.g., only one of the two or more reporters (e.g., only one of the first and second reporters) is repressed or increased from a basal level upon culture with a given test compound, then that test compound may be identified as not stimulating or repressing the biological activity of interest and, instead, may be identified as interfering with the expression of one of the reporters. If the expression of all of the two or more reporters (e.g., both the first and second reporters) is repressed or increased from a basal level upon culture with a given test compound, then that test compound may be identified as stimulating or repressing the biological activity of interest. Accordingly, the methods may comprise pre-treating the cells with a compound (e.g., an agonist or antagonist) that provides expression of the reporters (e.g., at a basal level). Upon treatment with a test compound that modulates the biological activity of interest, detection of an increase or decrease in reporter expression may identify the compound as modulating the biological activity of interest.

In an embodiment, the method comprises identifying at least one test compound that modulates at least one of the expression and the activity of each reporter. The one or more identified test compounds may modulate a biological activity in the cells.

Another embodiment of the invention provides a method of screening a library of test compounds for ability to inhibit or antagonize a biological activity of interest, the method comprising: (a) introducing a nucleic acid into a population of cells, wherein (i) the nucleic acid comprises a nucleotide sequence encoding two or more reporters including a first reporter and a second reporter that is different from the first reporter, (ii) the nucleic acid further comprises a nucleotide sequence encoding a ribosomal skip peptide positioned between nucleotide sequences encoding the first and second reporters, and (iii) the first and second reporters are stoichiometrically co-expressed under control of a transcriptional regulatory element that is activated by stimulation of the biological activity of interest prior to adding test compounds; (b) dividing the cells of (a) into more than one sub-population; (c) culturing each sub-population of cells with a test compound from the library, wherein each sub-population is cultured with a different test compound from the library; (d) measuring expression of the first and second reporters in each cultured sub-population of cells; and (e) identifying at least one test compound inhibiting the biological activity of interest when both of the first and second reporters expression is decreased by the sub-population of cells that was cultured with the test compound.

Another embodiment of the invention provides a nucleic acid comprising a nucleotide sequence encoding (i) two or more reporters comprising a first reporter and a second reporter that is different from the first reporter; and (ii) one or more ribosomal skip sequences, wherein a ribosomal skip sequence is positioned between the first and second reporters, wherein the first and second reporters are stoichiometrically co-expressed from the nucleotide sequence. In an embodiment, the nucleic acid does not comprise a cytomegalovirus-immediate early (CMV-IE) promoter. In an embodiment, the nucleic acid does not comprise a TRE and/or promoter. In an embodiment, the nucleic acid further comprises a nucleotide sequence comprising a transcriptional regulatory element (TRE) and/or promoter, wherein each of the first and second reporters is operably linked to the TRE and/or promoter. The TRE and/or promoter may be chosen by the skilled artisan on the basis of, for example, the biological activity of interest. In an embodiment, the nucleic acid further comprises nucleotide sequences flanking a combination of the nucleotide sequences encoding the two or more reporters and one or more ribosomal skip sequences and, optionally, the TRE and/or promoter, wherein the flanking nucleotide sequences are homologous to a left and right arm of a target site in a genome of the population of cells. The TRE and/or promoter, the flanking nucleotide sequences, and the nucleotide sequence encoding the first reporter, second reporter, and ribosomal skip sequence may be as described herein with respect to other aspects of the invention.

In an embodiment of the invention, the nucleic acid further comprises nucleotide sequences encoding insertion sites that facilitate the insertion of any TRE and/or promoter of interest into the nucleic acid. Such nucleotide sequences may be any suitable insertion sites as described in the art. See, for example, Green et al., supra, and Ausubel et al., supra. Examples of nucleotide sequences encoding insertion sites may include, but are not limited to, any one or more of restriction sites, Cre/loxP, Flp/FRT, mutant lox and FRT sites.

Another embodiment of the invention provides a nucleic acid comprising a nucleotide sequence encoding two or more reporters that are each different from one another and that are all stably stoichiometrically co-expressed under the control of a single promoter, and a ribosomal skip sequence peptide positioned between each nucleotide sequence encoding a different reporter.

“Nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide.

The nucleic acids of an embodiment of the invention may be recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

A recombinant nucleic acid may be one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques, such as those described in Green et al., supra. The nucleic acids can be constructed based on chemical synthesis and/or enzymatic ligation reactions using procedures known in the art. See, for example, Green et al., supra, and Ausubel et al., supra. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from companies, such as Macromolecular Resources (Fort Collins, Colo.) and Synthegen (Houston, Tex.).

An embodiment of the invention also provides an isolated or purified nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein. Alternatively, the nucleotide sequence can comprise a nucleotide sequence which is degenerate to any of the sequences or a combination of degenerate sequences.

The nucleotide sequence which hybridizes under stringent conditions may hybridize under high stringency conditions. By “high stringency conditions” is meant that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand, and are particularly suitable for detecting expression of any of the inventive nucleic acids. It is generally appreciated that conditions can be rendered more stringent by the addition of increasing amounts of formamide.

In an embodiment, the nucleic acids of the invention can be incorporated into a recombinant expression vector. In this regard, an embodiment of the invention provides recombinant expression vectors comprising any of the nucleic acids of the invention. For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the invention are not naturally-occurring as a whole. However, parts of the vectors can be naturally-occurring. The inventive recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring or non-naturally-occurring internucleotide linkages, or both types of linkages. Preferably, the non-naturally occurring or altered nucleotides or internucleotide linkages do not hinder the transcription or replication of the vector.

In an embodiment, the recombinant expression vector of the invention can be any suitable recombinant expression vector, and can be used to transform or transfect any suitable host cell. Suitable vectors include those designed for propagation and expansion or for expression or both, such as plasmids and viruses. The vector can be selected from the group consisting of the pUC series (Fermentas Life Sciences, Glen Burnie, Md.), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λGT10, λGT11, λZapII (Stratagene), λEMBL4, and λNM1149, also can be used. Examples of plant expression vectors include pBI01, pBI101.2, pBI101.3, pBI121 and pBIN19 (Clontech). Examples of animal expression vectors include pEUK-Cl, pMAM, and pMAMneo (Clontech). The recombinant expression vector may be a viral vector, e.g., a retroviral vector.

In an embodiment, the recombinant expression vectors of the invention can be prepared using standard recombinant DNA techniques described in, for example, Green et al., supra, and Ausubel et al., supra. Constructs of expression vectors, which are circular or linear, can be prepared to contain a replication system functional in a prokaryotic or eukaryotic host cell. Replication systems can be derived, e.g., from ColE1, 2μ plasmid, λ, SV40, bovine papilloma virus, and the like.

The recombinant expression vector may comprise additional regulatory sequences in addition to the TRE and/or promoters described herein, such as transcription and translation initiation and termination codons, which are specific to the type of host cell (e.g., bacterium, fungus, plant, or animal) into which the vector is to be introduced, as appropriate, and taking into consideration whether the vector is DNA- or RNA-based.

The recombinant expression vector can include one or more marker genes, which allow for selection of transformed or transfected host cells. Marker genes include biocide resistance, e.g., resistance to antibiotics, heavy metals, etc., complementation in an auxotrophic host to provide prototrophy, and the like. Suitable marker genes for the inventive expression vectors include, for instance, neomycin/G418 resistance genes, hygromycin resistance genes, histidinol resistance genes, tetracycline resistance genes, and ampicillin resistance genes.

An embodiment of the invention provides a virus comprising any of the nucleic acids described herein. The virus may be useful for infecting cells with any of the nucleic acids described herein and may, advantageously, provide for efficient transfection of cells.

An embodiment of the invention further provides a host cell comprising any of the recombinant expression vectors described herein. As used herein, the term “host cell” refers to any type of cell that can contain the inventive recombinant expression vector. The host cell can be any of the cells described herein with respect to other aspects of the invention. For purposes of amplifying or replicating the recombinant expression vector, the host cell may be a prokaryotic cell, e.g., a DH5α cell. For purposes of providing a cell-based assay, the host cell may be a mammalian cell. Preferably, the host cell is a human cell.

Also provided by an embodiment of the invention is a population of cells comprising at least one host cell described herein. The population of cells can be a heterogeneous population comprising the host cell comprising any of the recombinant expression vectors described, in addition to at least one other cell, e.g., a host cell which does not comprise any of the recombinant expression vectors. Alternatively, the population of cells can be a substantially homogeneous population, in which the population comprises mainly of host cells (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one embodiment of the invention, the population of cells is a clonal population comprising host cells comprising a recombinant expression vector as described herein.

The nucleic acids, recombinant expression vectors, and host cells (including populations thereof) can be isolated and/or purified. The teem “isolated” as used herein means having been removed from its natural environment. The term “purified” or “isolated” does not require absolute purity or isolation; rather, it is intended as a relative term. Thus, for example, a purified (or isolated) host cell preparation is one in which the host cell is more pure than cells in their natural environment within the body. Such host cells may be produced, for example, by standard purification techniques. In some embodiments, a preparation of a host cell is purified such that the host cell represents at least about 50%, for example at least about 70%, of the total cell content of the preparation. For example, the purity can be at least about 50%, can be greater than about 60%, about 70% or about 80%, or can be about 100%.

It is contemplated that the inventive cell-based assay materials may also be useful for methods of diagnosing a subject as having a condition. In this regard, another embodiment of the invention provides a method of diagnosing a subject as having a condition, the method comprising: (a) obtaining a sample from the subject, wherein the sample is suspected of containing an analyte associated with the condition; (b) introducing a nucleic acid into a population of cells, wherein (i) the nucleic acid comprises a nucleotide sequence encoding two or more reporters comprising a first reporter and a second reporter that is different from the first reporter, and (ii) the first and second reporters are stoichiometrically co-expressed under control of a transcriptional regulatory element that is activated or repressed in the presence of the analyte; (c) culturing the cells with the sample suspected of containing the analyte; (d) measuring expression of the first and second reporters by the cultured cells; and (e) diagnosing the patient as having the condition when both of the first and second reporters are expressed by the cultured cells or when a basal level of expression of both of the first and second reporters is repressed or increased in the sub-population of cells that is cultured with the test compound.

The method may comprise obtaining a sample from a subject, wherein the sample is suspected of containing an analyte associated with the condition. The subject referred to herein can be any subject. The subject may be a mammal. As used herein, the term “mammal” refers to any mammal, including, but not limited to, mammals of the order Rodentia, such as mice and hamsters, and mammals of the order Logomorpha, such as rabbits. The mammals may be from the order Carnivora, including Felines (cats) and Canines (dogs). The mammals may be from the order Artiodactyla, including Bovines (cows) and Swines (pigs) or of the order Perssodactyla, including Equines (horses). The mammals may be of the order Primates, Ceboids, or Simoids (monkeys) or of the order Anthropoids (humans and apes). Preferably, the mammal is a human.

The sample may be any sample obtained from the body of the subject. The sample may be, for example, blood, urine, saliva, tissue, or cells. The sample comprising cells can be a sample comprising whole cells, lysates thereof, or a fraction of the whole cell lysates, e.g., a nuclear or cytoplasmic fraction, a whole protein fraction, or a nucleic acid fraction. If the sample comprises whole cells, the cells can be any cells of the host, e.g., the cells of any organ or tissue, including blood cells or endothelial cells.

The analyte may be any molecule or chemical species the presence of which in the sample is associated with the existence of a given condition in the subject. In an embodiment of the invention, the analyte may be any of a metabolite, a hormone, a protein, DNA, RNA, a lipid, an antibody, a virus, a small organic molecule, a carbohydrate, and a toxin. In another embodiment of the invention, the analyte may be any of a lipoprotein, a low-density lipid (LDL), a high-density lipid (HDL), a cytokine, IL-6, C-reactive protein (CRP), N-terminal pro-brain natriuretic peptide (NT-proBNP), glycated hemoglobin, gelsolin, copeptin, thyroid-stimulating hormone (TSH), anti-thyroid peroxidase (TPO) antibody, carcinoembryonic antigen (CEA), alpha-fetoprotein (AFP), cancer antigen (CA) 125, CA 19-9, CA 27-29, beta-human chorionic gonadotropin (HCG), CA 15-3, calretinin, carcinoembryonic antigen, CD34, CD99, CD117, chromog, ranin, cytokeratin, desmin, epithelial membrane protein (EMA), factor VIII, CD31, FL1, glial fibrillary acidic protein (GFAP), gross cystic disease fluid protein (GCDFP-15), HMB-45, inhibin, keratin, PTPRC (CD45), MART-1 (Melan-A), Myo D1, muscle-specific actin (MSA), neuron-specific enolase (NSE), placental alkaline phosphatase (PLAP), prostate-specific antigen (PSA), 5100 protein, smooth muscle actin (SMA), synaptophysin, thyroglobulin, thyroid transcription factor-1, tumor M2-PK, and vimentin.

The condition may be any condition. In an embodiment, the condition may be cancer. The cancer can be any cancer, including any of acute lymphocytic cancer, acute myeloid leukemia, alveolar rhabdomyosarcoma, bladder cancer, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, fibrosarcoma, gastrointestinal carcinoid tumor, Hodgkin lymphoma, hypopharynx cancer, kidney cancer, larynx cancer, leukemia, liquid tumors, liver cancer, lung cancer, lymphoma, malignant mesothelioma, mastocytoma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer, skin cancer, small intestine cancer, soft tissue cancer, solid tumors, stomach cancer, testicular cancer, thyroid cancer, ureter cancer, and urinary bladder cancer.

In another embodiment, the condition is selected from the group consisting of thyroid disease; sepsis; cardiovascular disease; asthma; lung fibrosis; bronchitis; respiratory infections; respiratory distress syndrome; obstructive pulmonary disease; allergic diseases; multiple sclerosis; infections of the brain or nervous system; dermatitis; psoriasis; skin infections; gastroenteritis; colitis; Crohn's disease; cystic fibrosis; celiac disease; inflammatory bowel disease; intestinal infections; conjunctivitis; uveitis; infections of the eye; kidney infections; autoimmune kidney disease; diabetic nephropathy; cachexia; coronary restenosis; sinusitis, cystitis; urethritis; serositis; uremic pericarditis; cholecystis; vaginitis; drug reactions; hepatitis; pelvic inflammatory disease; lymphoma; multiple myeloma; vitiligo; alopecia; Addison's disease; Hashimoto's disease; Graves disease; atrophic gastritis/pernicious anemia; acquired hypogonadism/infertility; hypoparathyroidism; multiple sclerosis; Myasthenia gravis; Coombs positive hemolytic anemia; systemic lupus erthymatosis; Sjogren's syndrome, and diabetes.

In an embodiment, the condition is a viral disease. The viral disease may be caused by any virus. In an embodiment of the invention, the viral disease is caused by a virus selected from the group consisting of herpes viruses, pox viruses, hepadnaviruses, papilloma viruses, adenoviruses, coronoviruses, orthomyxoviruses, paramyxoviruses, flaviviruses, and caliciviruses. In a preferred embodiment, the viral disease is caused by a virus selected from the group consisting of pneumonia virus of mice (PVM), respiratory syncytial virus (RSV), influenza virus, herpes simplex virus, Epstein-Barr virus, varicella virus, cytomegalovirus, hepatitis A virus, hepatitis B virus, hepatitis C virus, human T-lymphotropic virus, calicivirus, adenovirus, and Arena virus.

The viral disease may be any viral disease affecting any part of the body. In an embodiment of the invention, the viral disease is selected from the group consisting of influenza, pneumonia, herpes, hepatitis, hepatitis A, hepatitis B, hepatitis C, chronic fatigue syndrome, sudden acute respiratory syndrome (SARS), gastroenteritis, enteritis, carditis, encephalitis, bronchiolitis, respiratory papillomatosis, meningitis, and mononucleosis, HIV, hemorrhagic fever viruses such as Ebola, Marburg, Lassa, and Hanta virus.

In an embodiment, when the condition is cardiovascular disease, the analyte may be any of a lipoprotein, LDL, a HDL, a cytokine, and IL-6. In another embodiment, when the condition is sepsis, the analyte may be any of a cytokine, CRP, gelsolin, and copeptin. In an embodiment, when the condition is thyroid disease, the analyte may be TSH and/or anti-TPO antibody. In an embodiment, when the condition is diabetes, the analyte may be C-peptide and/or glycated hemoglobin. In an embodiment, when the condition is cancer, the analyte may be any of CEA, AFP, CA 125, CA 19-9, CA 27-29, beta-HCG, CA 15-3, calretinin, carcinoembryonic antigen, CD34, CD99, CD117, chromogranin, cytokeratin, desmin, epithelial membrane protein (EMA), factor VIII, CD31, FL1, GFAP, GCDFP-15, HMB-45, inhibin, keratin, PTPRC (CD45), MART-1 (Melan-A), Myo D1, MSA, NSE, PLAP, PSA, S100 protein, SMA, synaptophysin, thyroglobulin, thyroid transcription factor-1, tumor M2-PK, and vimentin.

The method may comprise introducing a nucleic acid into a population of cells, wherein (i) the nucleic acid comprises a nucleotide sequence encoding two or more reporters comprising a first reporter and a second reporter that is different from the first reporter, and (ii) the first and second reporters are stoichiometrically co-expressed under control of a transcriptional regulatory element that is activated or repressed in the presence of the analyte. Introducing a nucleic acid into a population of cells may be carried out as described herein with respect to other aspects of the invention. The population of cells comprising the nucleic acid encoding the first and second reporters is distinct from a population of cells that is the sample obtained from the body of the subject.

The method may comprise culturing the cells comprising the nucleic acid encoding the two or more reporters with the sample suspected of containing the analyte and measuring expression of the two or more reporters by the cultured cells. Culturing the cells and measuring expression of the two or more reporters by the cultured cells may be carried out as described herein with respect to other aspects of the invention.

The method may comprise diagnosing the patient as having the condition when all of the two or more reporters are expressed by the cultured cells. If none of the two or more reporters (e.g., none of the first and second reporters) are expressed upon culture with a given sample, then that sample may be identified as not having the analyte, and the subject may be identified as not having the condition. If less than all of the reporters, e.g., only one of the two or more reporters (e.g., only one of the first and second reporters) are expressed upon culture with a given test compound, then that sample may be identified as not having the analyte and the subject may be identified as not having the condition. Instead, that sample may be identified as having an analyte that interferes with the expression of at least one of the reporters. If all of the two or more reporters (e.g., both the first and second reporters) are expressed upon culture with a given sample, then that sample may be identified as having the analyte and the subject may be identified as having the condition. The probability that an analyte will interact with all of the two or more reporters (e.g., both of the first and second reporters) instead of modulating the TRE and/or promoter is believed to be very low. Accordingly, the inventive methods and cell-based assay materials are believed to provide a more reliable measure of the presence of an analyte in the sample.

The method may comprise diagnosing the patient as having the condition when the expression of all of the two or more reporters by the cultured cells is repressed or increased. If the expression of none of the two or more reporters (e.g., none of the first and second reporters) is repressed or increased upon culture with a given sample, then that sample may be identified as not having the analyte, and the subject may be identified as not having the condition. If the expression of less than all of the reporters, e.g., only one of the two or more reporters (e.g., only one of the first and second reporters) is repressed or increased upon culture with a given test compound, then that sample may be identified as not having the analyte and the subject may be identified as not having the condition. Instead, that sample may be identified as having an analyte that interferes with the expression of at least one of the reporters. If the expression of all of the two or more reporters (e.g., both the first and second reporters) is repressed or increased upon culture with a given sample, then that sample may be identified as having the analyte and the subject may be identified as having the condition.

It is contemplated that one or more of the inventive cell-based assay materials may also be provided in a kit. In this regard, another embodiment of the invention provides a kit comprising: (a) a nucleic acid comprising a nucleotide sequence encoding (i) two or more reporters that are each different from one another and that are all stably stoichiometrically co-expressed under the control of a single promoter, and (ii) a ribosomal skip sequence peptide positioned between each nucleotide sequence encoding a different reporter; or a population of cells comprising the nucleic acid; and (b) a container for holding the nucleic acid or population of cells. Another embodiment of the invention provides a kit for screening a library of test compounds for ability to modulate a biological activity of interest or for diagnosing a subject as having a condition, the kit comprising: (a) (i) a nucleic acid comprising a nucleotide sequence encoding two or more reporters including a first reporter and a second reporter that is different from the first reporter and one or more ribosomal skip sequences, wherein a ribosomal skip sequence is positioned between the first and second reporters, wherein the first and second reporters are stoichiometrically co-expressed from the nucleotide sequence, and/or (ii) a population of cells comprising the nucleic acid; and (b) at least one container for holding the nucleic acid or population of cells. The nucleic acid, population of cells, reporters, and ribosomal skip sequence may be as described herein with respect to other aspects of the invention. In an embodiment of the invention, the kit comprises the population of cells comprising the nucleic acid, wherein the cells are mammalian cells.

The container(s) may be any container suitable for holding the nucleic acid or population of cells. For example, the container for holding the nucleic acid may be a tube and the container for holding the cells may be a gas-permeable bag or tube.

In an embodiment of the invention, the kit further comprises a cell culture plate. The cell culture plate may be any suitable cell culture plate for culturing the particular cells chosen and for detecting the detectable indicator of the presence or absence of the reporters. For example, the cell culture plate may be a multiwell plate.

The reporters may be as described herein with respect to other aspects of the invention. In an embodiment of the invention, the first reporter is firefly (FLuc) luciferase and the second reporter is Renilla (RLuc) luciferase.

In an embodiment of the invention, the nucleic acid of the kit comprises a TRE and/or promoter. The TRE and/or promoter may be chosen by the skilled artisan on the basis of, for example, the biological activity of interest. The TRE and/or promoter may be as described herein with respect to other aspects of the invention. In an embodiment of the invention, the two or more reporters are co-expressed under control of a transcriptional regulatory element (TRE) and/or promoter that is activated or repressed by modulation of the biological activity of interest, as described herein with respect to other aspects of the invention.

In another embodiment of the invention, the nucleic acid of the kit does not comprise a TRE and/or promoter. When the nucleic acid of the invention does not comprise a TRE and/or promoter, the TRE and/or promoter may be chosen by the skilled artisan on the basis of for example, the biological activity of interest and may be inserted into the nucleic acid as appropriate or the nucleic acid may be inserted into the genome of the population of cells so that the transcription of the reporters is under the control of a TRE and/or promoter that is endogenous to the population of cells, as described herein with respect to other aspects of the invention.

In an embodiment of the invention, the kit further comprises a first detection reagent that reacts with the first reporter to provide a detectable indicator of the presence or absence of the first reporter and a container for holding the first detection reagent. In another embodiment of the invention, the kit further comprises a second detection reagent that reacts with the second reporter to provide a detectable indicator of the presence or absence of the second reporter and a container for holding the second detection reagent. The containers for holding the two or more detection reagents may be any suitable container. The container may, for example, be a tube.

In an embodiment of the invention, the kit further comprises instructions for using the kit to perform any of the methods described herein.

In an embodiment of the invention, the kit further comprises one or more control compounds. The control compound may be used to calibrate the assay. For example, the control compound may be an inhibitor (such as, e.g., a ligand) of a reporter. The control compound may be used to quantitatively and/or qualitatively assess the basal level of reporter expression and/or to measure the output of the reporter upon encountering a test compound that interferes with the output of the reporter (e.g., by binding to the reporter).

In an embodiment of the invention in which the kit comprises a TRE and/or promoter associated with a particular biological activity of interest, the kit may further comprise known biological activity agonists and/or antagonists. The known biological activity agonists and/or antagonists may be used to assess the response of the assay and/or the sensitivity of the assay to molecules that are known to modulate or modulate the biological activity of interest.

The following examples further illustrate the invention but, of course, should not be construed as in any way limiting its scope.

### Example 1

This example demonstrates the ability of an assay using cells expressing FLuc-P2A-RLuc to discriminate between forskolin (FSK)-activated adenylyl cyclase signaling and signals mediated by inhibitors of FLuc and RLuc.

### Generation of FLuc-P2A-RLuc Constructs

The DNA oligonucleotides used are listed and depicted in Table 2. Nucleotides encoding Gly-Ser-Gly were added to the 5′ end of the high ‘cleavage’ efficiency 2A sequence from porcine teschovirus-1 (P2A) peptide (SEQ ID NO: 1). The pGL3-Control vector comprised an SV40 promoter operatively linked to a nucleotide sequence encoding FLuc. The pGL3-Control vector (Promega, Madison, Wis.) was used as the backbone to generate the SV40-driven FLuc-P2A-RLuc construct (pCI-6.20). First, oligonucleotides KC026 and KC027 (Integrated DNA Technologies, Skokie, Ill.) were used to remove the stop codon and add an EcoRI site by QUIKCHANGE II Site-Direct Mutagenesis Kit (Agilent Technologies, Wood Dale, Ill.) to create the construct pCI-6.17. Second, by using pRL-CMV vector (Promega) as the template, a Gly-Ser-Gly-P2A-RLuc fragment was generated by PCR using a 5′ primer (KC028) with an EcoRI site plus the Gly-Ser-Gly-P2A sequence and a 3′ primer (KC029) with an EcoRI site identical in reading frame to that found at the start codon of FLuc. The PCR product was then cut by EcoRI-HF (New England Biolabs, Ipswich, Mass.) and cloned into EcoRI site of pCI-6.17 to make the final pCI-6.20 construct. Accordingly, the pCl-6.20 construct comprised an SV40 promoter operably linked to a nucleotide sequence encoding FLuc, RLuc, and the P2A sequence positioned between FLuc and RLuc.

TABLE 2

| Oligo | SEQ ID | |
|---|---|---|
| Name | NO: | Sequence |
| KC026 | 15 | GAAGGGCGGAAAGATCGCCGTGGAATTCTAGAGTC |
| | | GGGGCGGCCGG |
| KC027 | 16 | CCGGCCGCCCCGACTCTAGAATTCCACGGCGATCT |
| | | TTCCGCCCTTC |
| KC028 | 17 | CCCGGCGTCTTGAATTCGGAAGCGGAGCTACTAAC |
| | | TTCAGCCTGCTGAAGCAGGCTGGAGACGTGGAGGA |
| | | GAACCCTGGACCTATGACTTCGAAAGTTTATGATC |
| | | CAGAAC |
| KC029 | 18 | CCCGGCGTCTTGAATTCTTATTGTTCATTTTTGAG |
| | | AACTCGCACAACG |
| KC030 | 19 | AGCTTGCTCGAGATCTGCGATCTAAGAGCCTGACGT |
| | | CAGAGAGCCTGACGTCAGAGAGCCTGACGTCAGAGA |
| | | GCCTGACGTCAGAGGAATTCAGACACTAGAGGGTAT |
| | | ATAATGGAAGCTCGACTTCCAGCTTGGCATTCCGGT |
| | | ACTGTTGGTAAAGA |
| KC031 | 20 | AGCTTAACTTTACCAACAGTACCGGAATGCCAAGC |
| | | TGGAAGTCGAGCTTCCATTATATACCCTCTAGTGT |
| | | CTGAATTCCTCTGACGTCAGGCTCTCTGACGTCAG |
| | | GCTCTCTGACGTCAGGCTCTCTGACGTCAGGCTCT |
| | | TAGATCGCAGATCTCGAGCA |

To create the 4XCRE-driven FLuc-P2A-RLuc construct (pCI-6.24), the promoterless FLuc-P2A-RLuc construct (pCI-6.22) was first generated using the pGL3-Enhancer vector (Promega) as the backbone. pCI-6.22 was made in exactly the same way as pCI-6.20 was made as described above. The oligonucleotides KC030 and KC031 containing 4XCRE plus minimal promoter sequences and HindIII sites at both ends were annealed and cloned into the HindIII site of pCI-6.22. The resulting construct was termed pCI-6.24. Accordingly, the pCl construct comprised 4XCRE (one CRE comprising SEQ ID NO: 13) operatively linked to a nucleotide sequence encoding FLuc, RLuc, and the P2A sequence positioned between FLuc and RLuc (SEQ ID NO: 3).

### Cell Culture and Transfection

The GripTite 293 MSR cell line was obtained from Life Technologies Corporation (Carlsbad, Calif.). Cells were maintained in DMEM-GLUTAMAX media (Life Technologies) supplemented with 10% fetal bovine serum (Life Technologies), 100 units/ml Penicillin and 100 μg/ml Streptomycin (Life Technologies). Transient transfection of plasmids into GRIPTITE 293 MSR cells (Life Technologies) was performed using LIPOFECTAMINE 2000 transfection reagent (Life Technologies) according to the manufacturer's instructions.

### Sequential Single-Well FLuc-RLuc Reporter Assay and Compound Test

This protocol measures bioluminescence derived from both FLuc and RLuc expression from a single assay. The stepwise protocol is provided in Table 3. Purified DNA constructs pGL3-Control and pCI-6.20 were co-transfected with p3XFLAG-CMV-7-BAP control plasmid (Sigma, St. Louis, Mo.) into GRIPTITE 293 MSR cells (Life Technologies). Sixteen hours after transfection, cells were trypsinized and then dispensed at 2,000 cells/20 μL/well in 384-well tissue culture treated white/solid bottom plates (Greiner Bio-One North America, Monroe, N.C.). The assay plates were incubated at 37° C. for 10 hours before adding the DUAL-GLO detection reagent (Promega). Luminescence from luciferase activity was detected by using a VIEWLUX plate reader (PerkinElmer, Waltham, Mass.).

TABLE 3
Sequential single-well FLuc-RLuc reporter assay (384- or 1536-well plate format)

| Step | Parameter | Value | | Description |
|---|---|---|---|---|
| 1 | Reagent | 20 μL or 4 μL | | ~2000/~500 cells into white/solid bottom plates |
| 2 | Incubation time | 1 | hour | 37° C. cell incubator |
| 3 | Compounds | 5 μL or 25 nL | | Pipette or Pin tool delivery |
| 4 | Incubation time | 10 | hours | 37° C. cell incubator |
| 5 | Reagent | 20 μL or 3.5 μL | | DUAL-GLO luciferase reagent, as per |
| | | | | manufacturer's instructions |
| 6 | Time | 10 | minutes | Cell lysis |
| 7 | Assay read 1 | 550-570 | nm | VIEWLUX plate reader |
| 8 | Reagent | 20 μL or 3.5 μL | | DUAL-GLO STOP & GLO reagent |
| 9 | Time | 10 | minutes | — |
| 10 | Assay read 2 | 550-570 | nm | VIEWLUX plate reader |

For the compound test, forskolin, PTC124 and BTS were prepared in a 24-point intraplate titration format and pre-diluted in the cell culture medium. Purified pCI-6.24 construct was transfected into GRIPTITE 293 MSR cells (Life Technologies). Sixteen hours post transfection, cells were trypsinized and then dispensed at 2,000 cells/15 μL/well in 384-well tissue culture treated white/solid bottom plates (Greiner Bio-One North America). Five μL of pre-diluted compound was transferred into assay plates, resulting in a final concentration ranging from 0.027 nM to 227 μM (forskolin) and 0.011 nM to 91 μM (PTC124 and BTS). The assay plates were incubated at 37° C. for 10 hours. FLuc and RLuc activities were then detected using DUAL-GLO reagent (Promega) and a VIEWLUX plate reader (PerkinElmer). Concentration-response curves and concentrations of half-maximal activity (EC50) for each compound were generated by using PRISM 4 software (GraphPad Software, Inc., La Jolla, Calif.).

### Preparation of Whole-Cell Extracts and Western Blot Analysis

Cells were rinsed with phosphate-buffered saline (PBS) (Life Technologies) and lysed in iced-cold M-PER mammalian protein extraction reagent (Thermo Scientific, Hanover Park, Ill.) supplemented with complete MINI protease inhibitor cocktail tablet (Roche Basel, Switzerland) 24 hours post-transfection. Each lysate was subject to SDS-polyacrylamide gradient gel (4-12% NUPAGE SDS-PAGE Gel System, Life Technologies) electrophoresis and transferred to PVDF membrane (Life Technologies). For Western blot analysis, the primary antibodies used were goat polyclonal anti-FLuc (1:1000, Promega), mouse monoclonal 5B11.2 anti-RLuc (1:1000, Millipore, Billerica, Mass.), rabbit polyclonal anti-2A peptide (1:1000, Millipore), mouse monoclonal anti-α-actin (1:1000, Sigma), and HRP-conjugated mouse monoclonal M2 anti-FLAG (1:4000, Sigma). Secondary antibodies were goat anti-mouse IgG-HRP (1:2000, Santa Cruz Biotechnology, Santa Cruz Calif.), donkey anti-goat IgG-HRP (1:2000, Santa Cruz Biotechnology), and goat anti-rabbit IgG-HRP (1:2000, Santa Cruz Biotechnology). The bound antibodies were detected using NOVEX ECL chemiluminescent substrate reagent kit (Life Technologies) and visualized by CHEMIDOC XRS+System (Bio-Rad, Des Plaines, Ill.).

LOPAC1280 qHTS Screening

The coincident biocircuit encoding FLuc and RLuc driven by a CRE array was used to identify compounds capable of eliciting an agonistic response in a HEK293 cell line derivative using quantitative high throughput screening HTS (qHTS). qHTS measures the pharmacological activity of each library compound by determining concentration response profiles of all library members (Inglese et al., Proc. Natl. Acad. Sci. USA, 103(31): 11473-78 (2006)). This was accomplished here as follows: purified DNA construct pCI-6.24 was transiently transfected into GRIPTITE 293 MSR cells (Life Technologies). Sixteen hours after transfection, cells were trypsinized and then dispensed at 500 cells/4 μL/well in 1,536-well tissue culture treated white/solid bottom plates (Greiner Bio-One North America) using a multidrop combi dispenser (Thermo Fisher Scientific). Compounds from the Library of Pharmacological Active Compounds (LOPAC), obtained from Sigma, were prepared as interplate titrations of seven dilutions (Yasgar et al., JALA Charlottesv. Va., 13(2): 79-89 (2008)). Twenty-three nL of compound from LOPAC was pin-transferred into the assay plates by a pin tool array (V&P Scientific, San Diego, Calif.) (Cleveland et al., Assay Drug Dev. Technol., 3(2): 213-225 (2005)) manipulated by an automated pin transfer station (Kalypsys, San Diego, Calif.) (Michael et al., Assay Drug Dev. Technol., 6(5): 637-57 (2008)). This resulted in a 174-fold dilution and the final compound concentration in the 4 μL assay ranged from ˜4 nM to 57 μM. The assay plates were incubated at 37° C. for 10 hours before adding the DUAL-GLO detection reagent (3.5 μL+3.5 μL for each well) (Promega). Luminescence from luciferase activity was detected by using VIEWLUX (PerkinElmer). Each experimental plate contained forskolin as a positive control and DMSO as a negative control. Percentage activity was defined as the percentage signal relative to forskolin (100%) and DMSO (0%). The assay performed well with signal-to-background ratios (S/B) of 3.37 for FLuc and 4.30 for RLuc, with additional parameters as set forth in Table 4.

TABLE 4

| | | | | | Intraplate | |
|---|---|---|---|---|---|---|
| | | | | | Forskolin | |
| | Assay | | | | Control (μM) | |
| Readout | Format | Z′ factor | S:B ratio | CV | Mean | s.d. |
| FLuc | 1536 | 0.40 | 3.37 | 23.87 | 0.86 | 0.36 |
| RLuc | interplate | 0.45 | 4.30 | 19.66 | 0.88 | 0.43 |

### FLuc and RLuc Enzymatic Assays

To determine compound potency against purified luciferase enzymes, 3 μL of luciferase substrate was dispensed to each well of 1536-well white/solid bottom plates (Greiner Bio-One North America) using the BioRaptor FRD (Beckman Coulter, Fullerton, Calif.), for a final concentration of 5 μM coelenterazine-H (Promega) or 10 μM D-luciferin (Sigma) and 10 μM ATP. Twenty-three nL of compounds were transferred using a 1536-pin tool (Wako, Richmond, Va.) into assay wells, resulting in final concentrations ranging from ˜3 nM to 57 μM with 11 titration points. One 1 μL of purified luciferase was dispensed into each well for a final concentration of 10 nM P. pyralis (FLuc) or 1 nM Renilla luciferase (RLuc). The bioluminescence outputs were measured by an ENVISION reader (PerkinElmer).

The function of a preliminary biocircuit design was confirmed by stoichiometric co-expression of the unrelated bioluminescent reporters, firefly (FLuc) and Renilla (RLuc) luciferase employing “ribosome skip” facilitated by the short P2A peptide (Inglese et al., Proc. Natl. Acad. Sci. USA, 103(31): 11473-78 (2006)) in a HEK293 cell. FLuc and RLuc are both sensitive reporters with generally short half-lives and use different substrates and mechanisms to produce light.

Western blot analysis showed the efficient expression of individual reporters, with little detectable fusion product, which would indicate poor ribosome skipping. Co-transfection of 3XFLAG-BAP demonstrated that the transfection efficiency was similar.

Bioluminescent output from mono FLuc reporter and co-expressed FLuc and RLuc was also measured. The results are shown in FIGS. 1A and 1B. As shown in FIGS. 1A and 1B, cells expressing the FLuc-P2A-RLuc dual reporter (pCI-6.20) produced bioluminescent output for both RLuc and FLuc.

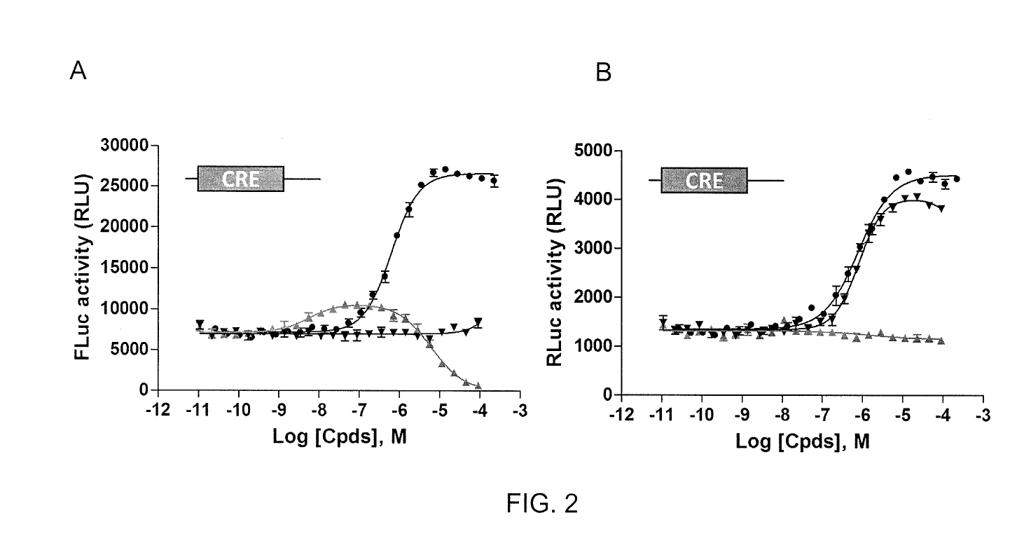

The accurate discrimination of forskolin (FSK)-activated adenylyl cyclase signaling was demonstrated through the cAMP-response element (CRE) from signals mediated by the known FLuc and RLuc stabilizers, PTC124 and BTS, respectively (FIGS. 2A-2B). PTC124 and BTS are inhibitors of FLuc and RLuc, respectively, and act to increase the activity of the reporters by stabilizing their cellular half-life relative to non-treated control. This experiment was repeated with cells transfected with the pCl-6.20 construct, which encoded FLuc-P2A-RLuc under the control of the SV40 response element. FSK was inactive in experiments where reporter expression was driven by the SV40 promoter, only displaying activity when the biocircuit was under control of 4XCRE.

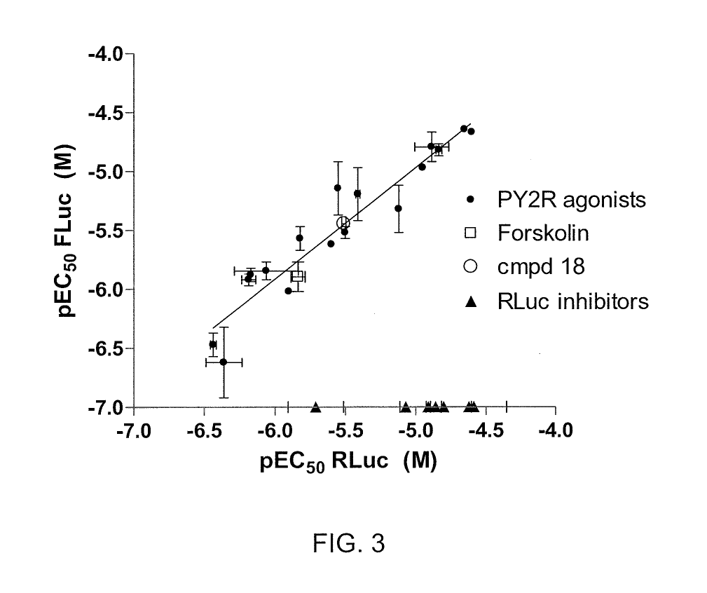

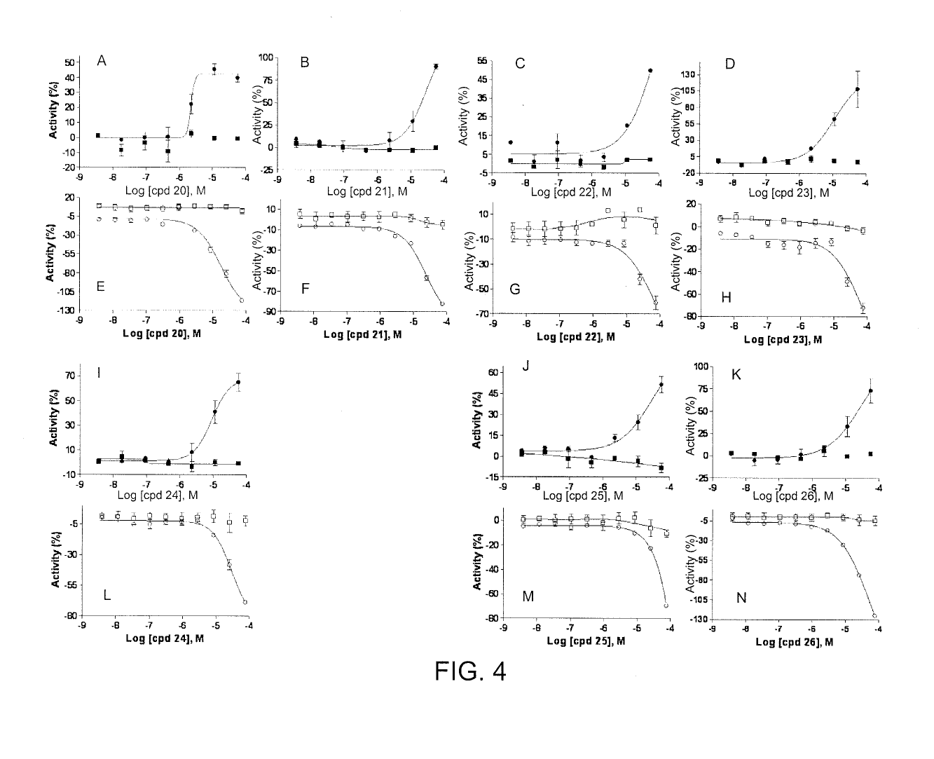

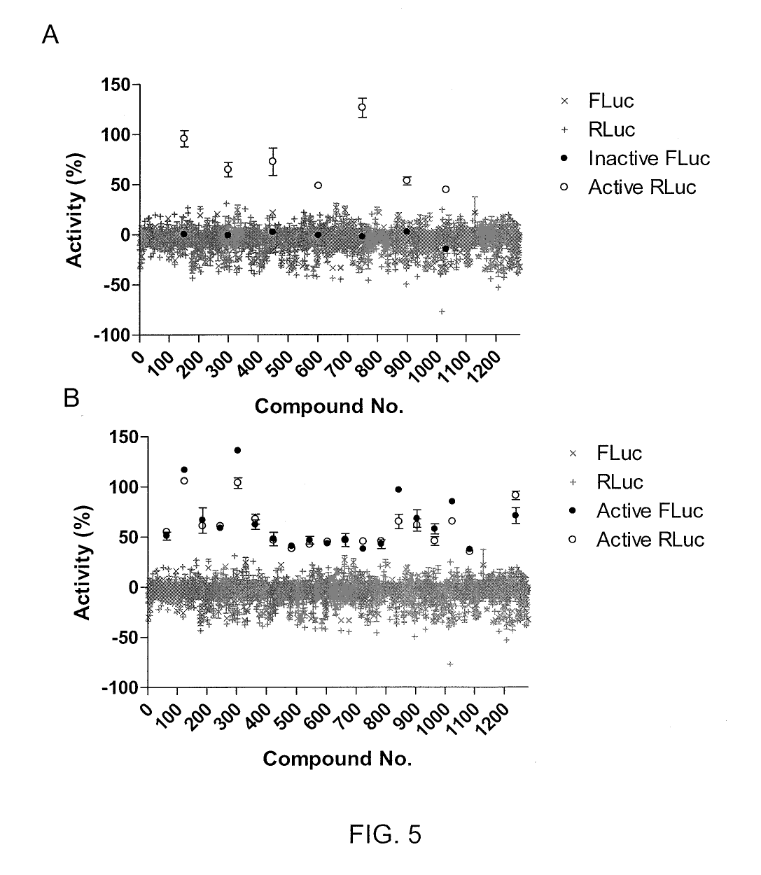

Using the LOPAC1280 chemical library, a quantitative HTS (qHTS) experiment was conducted in which full titrations of each compound were tested to identify potentiators of the CREB pathway. The screen revealed, for example, coincident FLuc and RLuc signal outputs for 17 adenosine analog agonists of endogenous purinergic 2Y and one muscarinic receptor agonist (Arecaidine propargyl ester, cpd 18) known to signal through G-proteins in this cell type, and the adenyl cyclase activator forskolin, cpd 19 (Table 5) Excellent correlation between the EC₅₀ values calculated from the orthogonal reporter outputs was observed (FIG. 3). Illustrating the phenomenon of reporter-dependent artifacts, five aryl sulfonamides and two aryl (vinyl) sulfanes (cpd 25-26) were identified that showed selective agonist activity for RLuc only (Table 6). These compounds share a similar core scaffold with two known RLuc inhibitors and selectively inhibit the enzymatic activity of RLuc over FLuc, thus tying these particular artifacts to the phenomenon of reporter stabilization (FIGS. 4A-4N). As shown in FIGS. 4A-4N, the cell based activation response mirrors the enzymatic inhibition on the respective reporter. Cross-section data analysis of the screen (FIGS. 5A-5B) also demonstrates how coincidence detection enhances the testing of compound libraries in single concentration format.

TABLE 5

| | | | | FLuc EC₅₀ | RLuc | |
|---|---|---|---|---|---|---|
| Category | cpd # | SID | LOPAC ID | (μM) | EC₅₀ (μM) | Ratio F/R |
| 1 | 13 | NCGC00025260-05 | Lopac-E-2397 | 0.30 | 0.54 | 0.56 |
| 1 | 5 | NCGC00093771-04 | Lopac-C-9901 | 16.94 | 25.12 | 0.67 |
| 1 | 10 | NCGC00024978-05 | Lopac-I-146 | 5.29 | 7.57 | 0.70 |
| 1 | 6 | NCGC00023909-06 | Lopac-C-8031 | 0.95 | 1.26 | 0.75 |
| 1 | 16 | NCGC00162286-02 | Lopac-N-7505 | 18.20 | 22.39 | 0.81 |
| 1 | 7 | NCGC00162105-02 | Lopac-G-5794 | 2.69 | 3.16 | 0.85 |
| 1 | 2 | NCGC00023481-04 | Lopac-P-108 | 12.73 | 14.62 | 0.87 |
| 1 | 15 | NCGC00162362-02 | Lopac-T-5515 | 2.39 | 2.51 | 0.95 |
| 1 | 4 | NCGC00025270-03 | Lopac-P-101 | 10.69 | 11.22 | 0.95 |
| 1 | 11 | NCGC00021540-06 | Lopac-C-5134 | 0.43 | 0.38 | 1.13 |
| 1 | 12 | NCGC00162241-04 | Lopac-M-5501 | 16.67 | 13.27 | 1.26 |
| 1 | 8 | NCGC00015017-05 | Lopac-A-202 | 1.45 | 0.93 | 1.56 |
| 1 | 14 | NCGC00025218-02 | Lopac-H-3288 | 2.69 | 1.51 | 1.78 |
| 1 | 3 | NCGC00015640-04 | Lopac-M-225 | 1.34 | 0.61 | 2.20 |
| 1 | 17 | NCGC00162130-02 | Lopac-C-145 | 11.50 | 3.89 | 2.96 |
| 1 | 1 | NCGC00162295-03 | Lopac-P-4532 | 9.37 | 2.82 | 3.32 |
| 1 | 9 | NCGC00162075-03 | Lopac-A-236 | 1.51 | 0.43 | 3.51 |
| 2 | 18 | NCGC00015006-04 | Lopac-A-140 | 3.59 | 3.09 | 1.16 |
| 3 | 19 | NCGC00015445-05 | Lopac-F-6886 | 1.32 | 1.47 | 0.90 |

| Category | cpd # | Sample Name | Description |
|---|---|---|---|
| 1 | 13 | 5′-N- | adenosine receptor agonist with equal |
| | | Ethylcarboxamidoadenosine | affinity at A₁ and A₂ receptors |
| 1 | 5 | N6-Cyclohexyladenosine | selective A₁ adenosine receptor agonist |
| 1 | 10 | IB-MECA | A₃ adenosine receptor agonist |
| 1 | 6 | N6-Cyclopentyladenosine | selective A₁ adenosine receptor agonist |
| 1 | 16 | NADPH tetrasodium | a ubiquitous cofactor and biological |
| | | | reducing agent |
| 1 | 7 | GR 79236X | A₁ adenosine receptor agonist |
| 1 | 2 | N6-Phenyladenosine | A₁ adenosine receptor agonist |
| 1 | 15 | Thio-NADP sodium | blocks nicotinate adenine dinucleotide |
| | | | phosphate (NAADP)-induced Ca²⁺ release |
| 1 | 4 | 2-Phenylaminoadenosine | selective A₂ adenosine receptor agonist |
| 1 | 11 | 2-Chloroadenosine | adenosine receptor agonist with selectivity |
| | | | for A₁ over A₂ |
| 1 | 12 | N6-Methyladenosine | selective A₁ adenosine receptor agonist |
| 1 | 8 | N6-2-(4- | non-selective A₃ adenosine receptor |
| | | Aminophenyl)ethyladenosine | agonist |
| 1 | 14 | HEMADO | selective A₃ adenosine receptor agonist |
| 1 | 3 | Metrifudil | adenosine receptor agonist which displays |
| | | | some selectivity for the A₂ receptor type |
| 1 | 17 | 2-Chloroadenosine | P2Y purinoceptor agonist |
| | | triphosphate tetrasodium | |
| 1 | 1 | R(−)-N6-(2- | A₁ adenosine receptor agonist |
| | | Phenylisopropyl)adenosine | |
| 1 | 9 | AB-MECA | A₃ adenosine receptor agonist |
| 2 | 18 | Arecaidine propargyl ester | muscarinic acetylcholine receptor agonist |
| | | hydrobromide (APE) | exhibiting slight selectivity for M₂ receptor |
| 3 | 19 | Forkskolin | adenylyl cyclase activator |

TABLE 6

| Class | Cpd # | SID | LOPAC ID | FLuc EC₅₀ (μM) | RLuc EC₅₀ (μM) |
|---|---|---|---|---|---|
| 1 | 20 | NCGC00015885-04 | Lopac-R-140 | N/A | 2.05 |
| 1 | 24 | NCGC00015380-12 | Lopac-D-9035 | N/A | 9.15 |
| 1 | 22 | NCGC00024555-06 | Lopac-A-1980 | N/A | 15.85 |
| 1 | 21 | NCGC00015379-04 | Lopac-D-8941 | N/A | 21.44 |
| 1 | 23 | NCGC00015467-16 | Lopac-G-0639 | N/A | 30.35 |
| 2 | 25 | NCGC00094462-03 | Lopac-U-120 | N/A | 8.49 |
| 2 | 26 | NCGC00015889-07 | Lopac-R-1402 | N/A | 12.00 |

| Class | Cpd # | Ratio F/R | Sample Name | Description |
|---|---|---|---|---|
| 1 | 20 | N/A | Ro 04-6790 hydrochloride | selective 5-HT₆ serotonin receptor |
| | | | | antagonist |
| 1 | 24 | N/A | Diazoxide | selective AMPA ionotropic |
| | | | | glutamate receptor agonist |
| 1 | 22 | N/A | A3 hydrochloride | selective estrogen receptor |
| | | | | modulator |
| 1 | 21 | N/A | 2,6-Difluoro-4-[2- | non-selective casein kinase (CK) |
| | | | (phenylsulfonylamino) | inhibitor |
| | | | ethylthio]phenoxyacetamide | |
| 1 | 23 | N/A | Glybenclamide | selective inhibitor of both MEK1 |
| | | | | and MEK2 |
| 2 | 25 | N/A | U0126 | selectively blocks ATP-sensitive |
| | | | | K⁺ channels |
| 2 | 26 | N/A | Raloxifene hydrochloride | selective ATP-sensitive K+ |
| | | | | channels activator |

It is concluded that coincidence reporter strategies rapidly discriminate compounds of relevant biological activity from those interfering with reporter function and stability using a single assay platform.

### Example 2

This example demonstrates the bioluminescent output of cells expressing a 4XCRE-driven FLuc-P2A-emGFP construct.

A 4XCRE-driven FLuc-P2A-emGFP construct was generated as follows. All DNA oligonucleotides used to generate this construct are listed and depicted in Table 7. Nucleotides encoding Gly-Ser-Gly were added to the 5′ end of the high ‘cleavage’ efficiency 2A sequence from porcine teschovirus-1 (P2A). First, pCI-6.24 was cut using the EcoRI site to remove the P2A-RLuc open reading frame (ORF). Second, by using VIVIDCOLORS pcDNA-6.2/C-emGFP-DEST vector (Life Technologies) as the template, a Gly-Ser-Gly-P2A-emGFP fragment was generated by PCR using a 5′ primer (KC040) with an EcoRI site plus the Gly-Ser-Gly-P2A sequence and a 3′ primer (KC041) with an EcoRI site identical in reading frame to that found at the start codon of emGFP. The PCR product was then cut by EcoRI-HF (New England Biolabs) and cloned into the EcoRI site of pCI-6.24 to make the final pCI-6.25 construct.

TABLE 7
Oligonucleotide sequences used in pCI-6.25
Construction

| Oligo | SEQ ID | |
|---|---|---|
| Name | NO: | Sequence |
| KC040 | 345 | CCCGGCGTCTTGAATTCGGAAGCGGAGCTACTAAC |
| | | TTCAGCCTGCTGAAGCAGGCTGGAGACGTGGAGGA |
| | | GAACCCTGGACCTATGGTGAGCAAGGGCGAGGAGC |
| | | TGTTC |
| KC041 | 346 | CCCGGCGTCTTGAATTCTTAGTACAGCTCGTCCAT |
| | | GCCGAGAGTGATC |

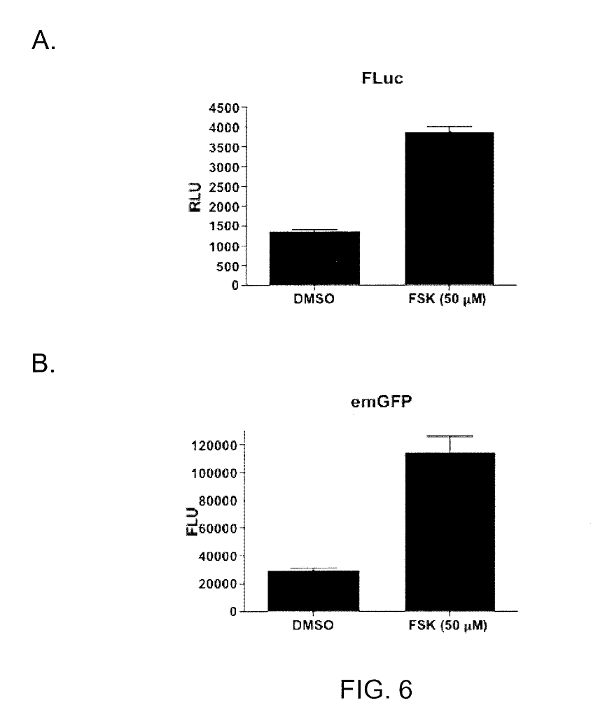

A sequential single-well FLuc-emGFP reporter assay and compound test was carried out as follows. This protocol measures bioluminescence derived from FLuc and fluorescence from emGFP expression from a single assay. Purified DNA constructs pCI-6.25 were transfected into GripTite 293 MSR cells (Life Technologies) and the cells were cultured as described in Example 1. Sixteen hours after transfection, cells were trypsinized and then dispensed at 2,000 cells/20 μL/well in 384-well tissue culture treated black/clear bottom plates (Aurora). After adding Forskolin (FSK) (Sigma) or control DMSO, the assay plates were incubated at 37° C. for 10 hours before measuring fluorescence from emGFP expression by ACUMEN high content imaging (TTP Labtech, Cambridge, UK). Then the ONE-GLO detection reagent (Promega) was added and the bioluminescence from luciferase activity was detected by using a VIEWLUX plate reader (PerkinElmer). The results are shown in FIGS. 6A-6B. As shown in FIGS. 6A-6B, cells transfected with 4XCRE-driven FLuc-P2A-emGFP constructs demonstrated greater RLU values when treated with forskolin as compared to those treated with DMSO.

### Example 3

This example demonstrates the bioluminescent output of cells expressing a 4XCRE-driven NLucP-P2A-emGFP construct.

A 4XCRE-driven NLucP-P2A-emGFP construct was generated as follows. All DNA oligonucleotides used to generate this construct are listed and depicted in Table 8. Nucleotides encoding Gly-Ser-Gly were added to the 5′ end of the high ‘cleavage’ efficiency 2A sequence from porcine teschovirus-1 (P2A). pCI-6.24 was partially digested using the NcoI and EcoRI sites to remove the FLuc ORF. Second, by using the pNL-1.2 vector (Promega) as the template, a NLucP fragment was generated by PCR using a 5′ primer (KC071) with an NcoI site and a 3′ primer (KC072) with an EcoRI site identical in reading frame to that found at the start codon of NLucP. The PCR product was then cut by NcoI/EcoRI-HF (New England Biolabs) and cloned into NcoI/EcoRI site of pCI-6.24 to make the final pCI-6.48 construct.

TABLE 8

| Oligo | SEQ ID | |
|---|---|---|
| name | NO: | Sequence |
| KC071 | 347 | CACCGG TACTGTTGGT AAAGCCACCATG G |
| KC072 | 348 | CCCCCCCGAATTCGACGTTGATGCGAGCTGAA |
| | | GCAC |

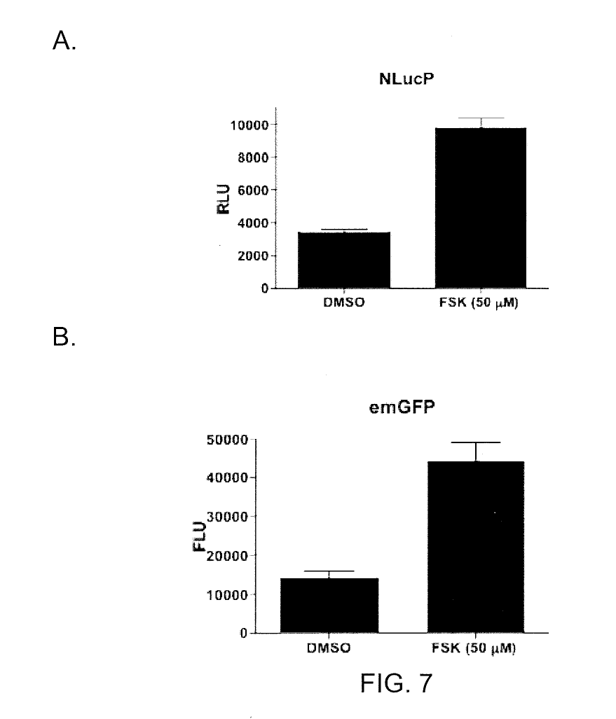

A sequential single-well NLuc-emGFP reporter assay and compound test was carried out as follows. This protocol measures bioluminescence derived from NLuc and fluorescence from emGFP expression from a single assay. Purified DNA constructs pCI-6.25 were transfected into GripTite 293 MSR cells (Life Technologies) and the cells were cultured as described in Example 1. Sixteen hours after transfection, cells were trypsinized and then dispensed at 2,000 cells/20 μL/well in 384-well tissue culture treated black/clear bottom plates (Aurora). After adding Forskolin (FSK) (Sigma) or control DMSO, the assay plates were incubated at 37° C. for 10 hours before measuring fluorescence from emGFP expression by Acumen (TTP Labtech). Then the ONE-GLO detection reagent (Promega) was added and the bioluminescence from luciferase activity was detected by using a VIEWLUX plate reader (PerkinElmer). The results are shown in FIGS. 7A and 7B. As shown in FIGS. 7A-7B, cells transfected with 4XCRE-driven NLucP-P2A-emGFP constructs demonstrated greater RLU values when treated with forskolin as compared to those treated with DMSO.

### Example 4

This example demonstrates the bioluminescent output of cells expressing a p53 RE-driven FLuc2P-P2A-NLucP construct.

p53 RE-driven FLuc2P-P2A-NLucP constructs were generated as follows. All DNA oligonucleotides used to generate this construct are listed and depicted in Table 9. Nucleotides encoding Gly-Ser-Gly were added to the 5′ end of the high ‘cleavage’ efficiency 2A sequence from porcine teschovirus-1 (P2A). First, the pGL-4.38 vector (Promega) was used as the backbone to generate the p53 RE-driven FLuc-P2A-NLuc construct (pCI-4.38). Oligonucleotides KC065 and KC066 (Integrated DNA Technologies) were used to remove the stop codon and add a SmaI site by QUIKCHANGE II Site-Direct Mutagenesis Kit (Agilent Technologies) to create the construct pCI-6.36. pCI-6.36 was digested with SmaI (New England Biolabs) and ligated with Frame B of GATEWAY Conversion System (Life Technologies) to make the GATEWAY pCI-5.08 vector. The LR reaction was then performed using the pCI-5.08 and pCI-1.09 vectord to make the final pCI-4.38 construct.

TABLE 9

| Oligo | SEQ ID | |
|---|---|---|
| name | NO: | Sequence |
| KC065 | 349 | GCCAGCGCCAGGATCAACGTCCCGGGCCGCGAC |
| | | TCTAGAG |
| KC066 | 350 | CTCTAGAGTCGCGGCCCGGGACGTTGATCCTGG |
| | | CGCTGGC |

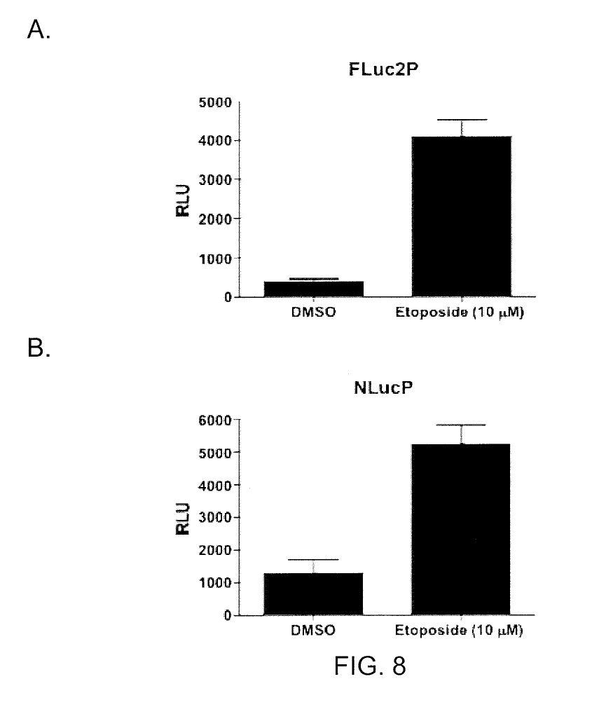

The FLuc2P-NLucP reporter assay and compound test was carried out as follows. This protocol measures bioluminescence derived from both FLuc2P and NLucP. The purified DNA construct pCI-4.38 was transfected into HEK293 cells and the cells were cultured as described in Example 1. Sixteen hours after transfection, the cells were trypsinized and then dispensed at 2,000 cells/20 μL/well into two 384-well tissue culture treated white/solid bottom plates (Greiner Bio-One North America). After adding Etoposide (Sigma) or control DMSO, the assay plates were incubated at 37° C. for 24 hours before adding the ONE-GLO or NANO-GLO detection reagents (Promega). Luminescence from luciferase activity was detected by using a VIEWLUX plate reader (PerkinElmer). The results are shown in FIGS. 8A and 8B. As shown in FIGS. 8A-8B, cells transfected with a p53 RE-driven FLuc2P-P2A-NLucP construct demonstrated greater RLU values when treated with etoposide as compared to those treated with DMSO.

### Example 5

This example demonstrates the bioluminescent output of cells expressing an ARE-driven FLuc2P-P2A-NLucP construct.

An ARE-driven FLuc2P-P2A-NLucP construct was generated as follows. All DNA oligonucleotides used to generate this construct are listed and depicted in Table 10. Nucleotides encoding Gly-Ser-Gly were added to the 5′ end of the high ‘cleavage’ efficiency 2A sequence from porcine teschovirus-1 (P2A). The pGL-4.37 vector (Promega) was used as the backbone to generate the ARE-driven FLuc-P2A-NLuc construct (pCI-4.37). First, oligonucleotides KC065 and KC066 (Integrated DNA Technologies) were used to remove the stop codon and add a SmaI site by QUIKCHANGE II Site-Direct Mutagenesis Kit (Agilent Technologies) to create the construct pCI-6.35. pCI-6.35 was digested with SmaI (New England Biolabs) and ligated with Frame B of GATEWAY Conversion System (Life Technologies) to make the GATEWAY pCI-5.07 vector. The LR reaction was then performed using pCI-5.07 and pCI-1.09 vector to make the final pCI-4.37 construct.

TABLE 10

| Oligo | SEQ ID | |
|---|---|---|
| name | NO: | Sequence |
| KC065 | 351 | GCCAGCGCCAGGATCAACGTCCCGGGCCGCGACT |
| | | CTAGAG |
| KC066 | 352 | CTCTAGAGTCGCGGCCCGGGACGTTGATCCTGGC |
| | | GCTGGC |

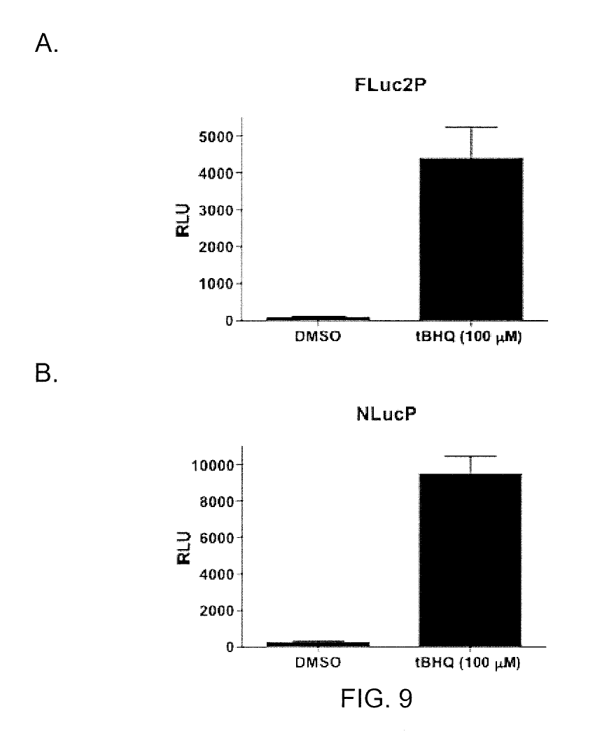

A FLuc2P-NLucP reporter assay and compound test was carried out as follows. This protocol measures bioluminescence derived from both FLuc2P and NLucP. A purified DNA construct pCI-4.37 was transfected into HEK293 cells. Sixteen hours after transfection, the cells were trypsinized and dispensed at 2,000 cells/20 μL/well into two 384-well tissue culture treated white/solid bottom plates (Greiner Bio-One North America). After adding tert-Butylhydroquinone (tBHQ) (Sigma) or control DMSO, the assay plates were incubated at 37° C. for 24 hours before adding the ONE-GLO or NANO-GLO detection reagents (Promega). Luminescence from luciferase activity was detected by using a VIEWLUX plate reader (PerkinElmer). The results are shown in FIGS. 9A-9B. As shown in FIGS. 9A and 9B, cells transfected with an ARE-driven FLuc2P-P2A-NLucP construct demonstrated greater RLU values when treated with tBHQ as compared to those treated with DMSO.

### Example 6

This example demonstrates the targeted placement of Fluc-P2A-NLucP into the PARK2 gene locus.

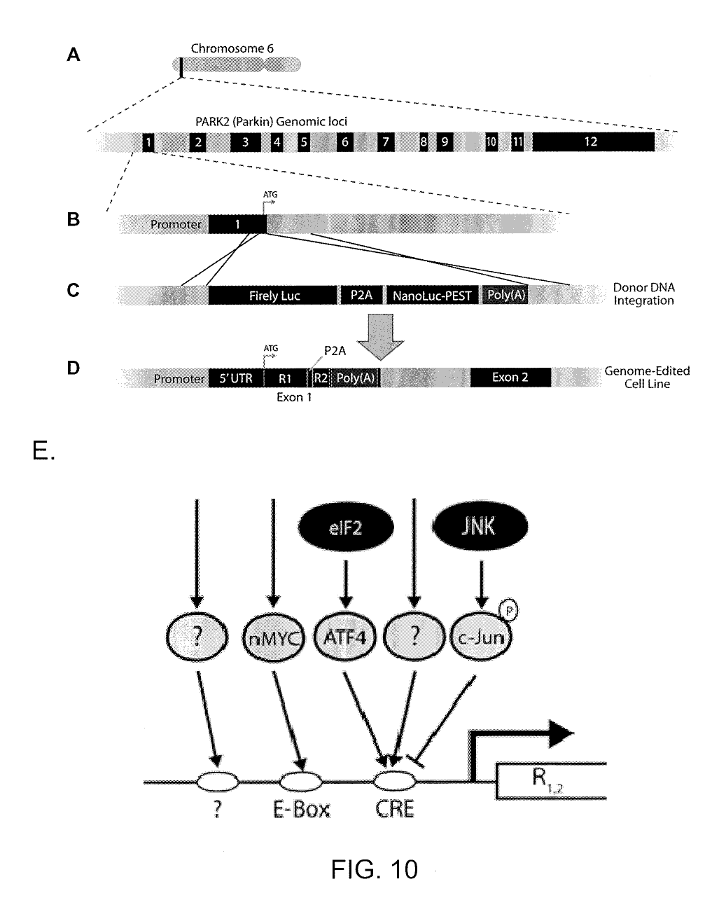

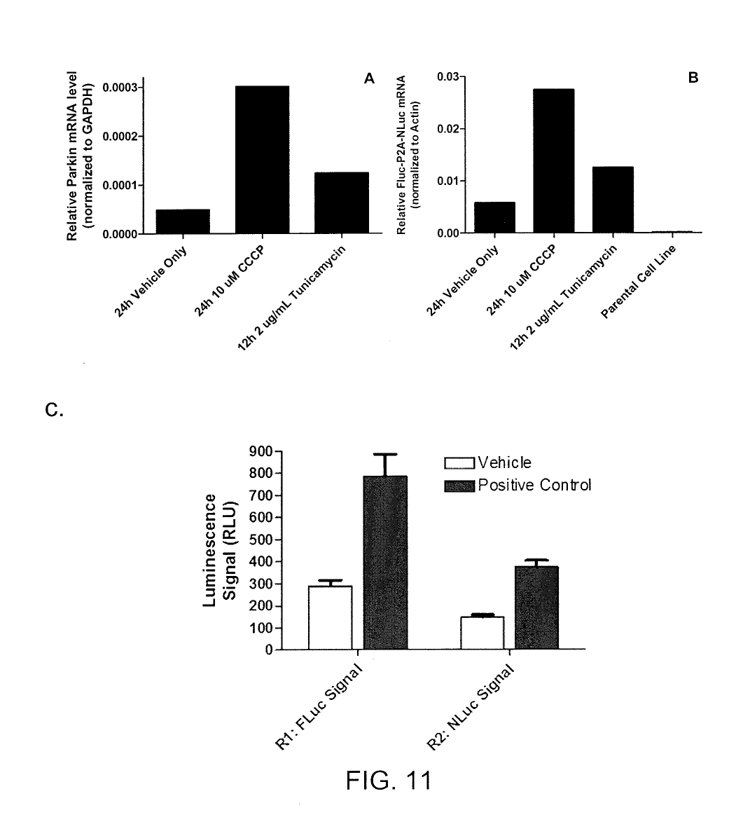

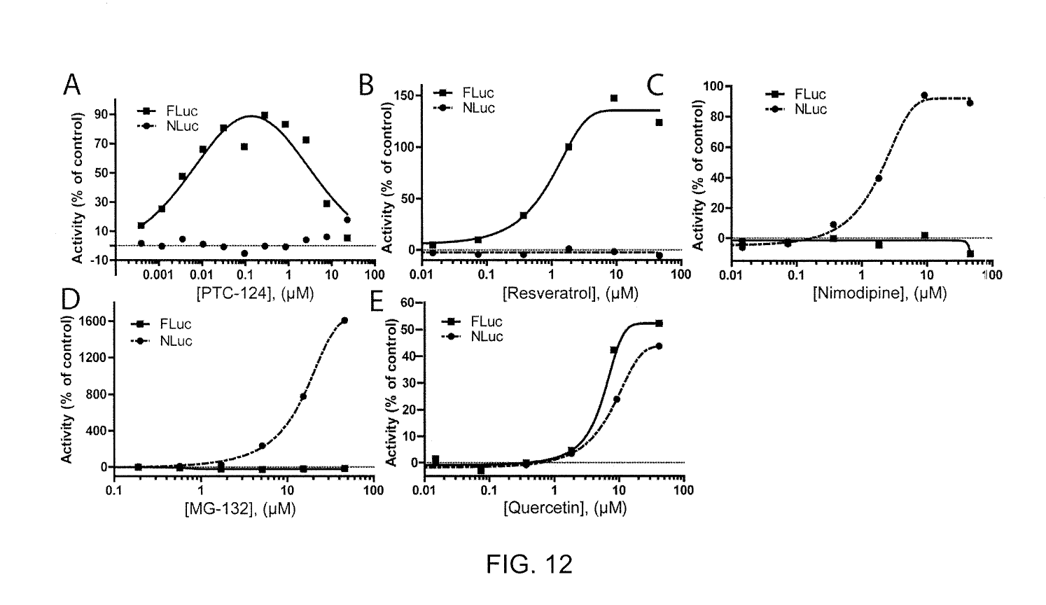

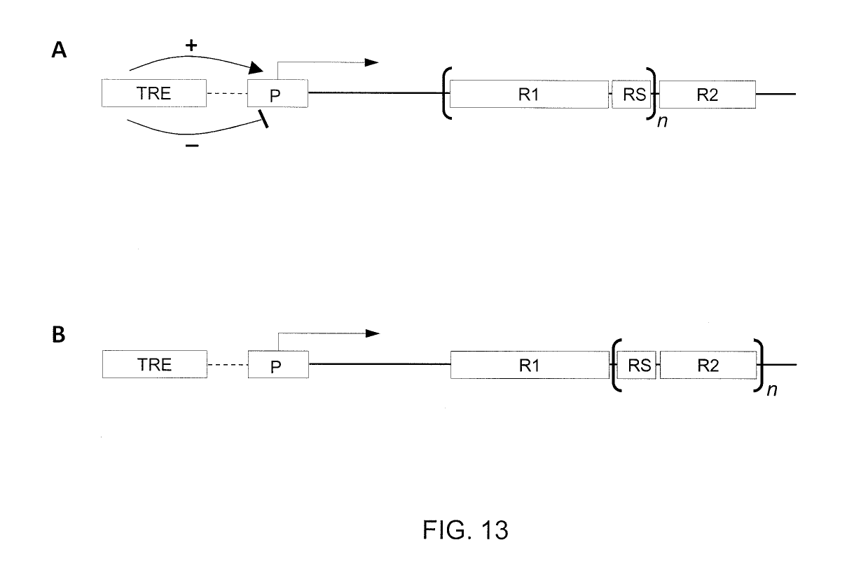

The targeting of a Fluc-P2A-NLucP coincidence reporter to specific gene locus allowed endogenous mechanisms of gene regulation of the PARK2 gene to be monitored using a coincidence reporter (FIG. 10E). The FLuc-P2A-NLucP coincidence reporter was targeted to the PARK2 gene locus on chromosome 6 using TALEN-mediated genome editing (FIGS. 10A-10D).

The cloning of the FLuc-P2A-NLuc construct and donor DNA was carried out as follows. To generate the Fluc-P2A-NLuc-PEST construct, the existing FLuc-P2A-RLuc construct (pCI-6.20) was PCR amplified as a linear fragment lacking the RLuc gene using primers flanking the RLuc gene (Primers: Forward 5′-GAATTCTAGAGTCGGGGC-3′ (SEQ ID NO: 353), and Reverse 5′-AGGTCCAGGGTTCTCCTC-3′ (SEQ ID NO: 354)). A PCR fragment encompassing the NanoLuc-PEST gene was also amplified from the pNL1.2 (Promega) vector with primers containing 15 base-pairs of homology to the target pCI vector fragment (Primers: Forward 5′-GAGAACCCTGGACCTATGGTCTTCACACTCGAAG-3′ (SEQ ID NO: 355), and Reverse 5′-CCGACTCTAGAATTCTTAGACGTTGATGCGAGC-3′ (SEQ ID NO: 356)). The NanoLuc-PEST gene PCR fragment was then joined with the pCI-6.20 PCR fragment using InFusion cloning (Clontech) according to manufacturer's protocols to reconstitute a circular plasmid. The resulting pCW-7 construct contained the FLuc-P2A-NLuc followed by a SV40 late poly(A) signal sequence. This entire cassette (Fluc-P2A-NLuc-PEST-PolyA) was PCR amplified (Primers: Forward 5′-ATGGAAGACGCCAAAAAC-3′ (SEQ ID NO: 357), and Reverse 5′-TCGATTTTACCACATTTGTAGAG3′ (SEQ ID NO: 358)) and transferred into a donor DNA vector between ˜1 kb segments of human genomic DNA sequence flanking the 5′ and 3′ of the PARK2 (Parkin) gene exon 1 by InFusion cloning. The PARK2 genomic sequence had been inserted into the pBluescript II SK (Addgene) donor plasmid as a complete ˜2 kb genomic fragment of the PARK2 gene (Homo sapiens chromosome 6, GRCh37.p10 Primary Assembly coordinate 67317052-67319214) by PCR amplification from human genomic DNA (Primers: Forward 5′-ATATCGAATTCTTTGCTGAGTGGGGCTAG-3′ (SEQ ID NO: 359), and Reverse 5′-CTAGTGGATCCCCACTGATGGGGAGAATG (SEQ ID NO: 360)) cloning into the donor vector EcoRI and BamHI restriction sites.

Construction of the Parkin coincidence reporter cell line by TALEN-mediated genome editing was carried out as follows. To generate a double-strand cleavage of the genomic DNA in the first codon of the PARK2 gene, constructs encoding transcription activator-like effector nuclease (TALEN) pairs (Right and Left) encoding components of the heterodimeric FokI nuclease were generated as described by Huang et al., Nature Biotechnology, 29: 699-700 (2011). The TALEN pair was designed to generate a double-strand cleavage at or near the first translation codon (ATG) within the Parkin gene. These constructs were transfected with Lipofectamine LTX (Life Technologies) into BE(2)-M17 cells (ATCC) (SEQ ID NO: 361) along with a GFP-expressing marker plasmid and the coincidence reporter donor plasmid. After 48 hours of incubation in a tissue culture incubator, GFP-positive cells were sorted by FACS analysis and single clones were isolated and expanded. Once sufficient cell populations for each clone were achieved, analysis of correct genomic insertion of the Fluc-P2A-NLuc-PEST-PolyA coincidence reporter cassette that replaced the “ATGATAG” (SEQ ID NO: 362) sequence at the 3′ end of the PARK2 gene exon 1 was ascertained by PCR and DNA sequencing of genomic DNA preparations (QIAGEN).

Final selection of clones for high throughput screening was then performed by selecting those that demonstrated a robust luciferase or gene transcription inductions after 24 hour treatment with 10 μM carbonyl cyanide m-chlorophenyl hydrazone and 2 ug/mL Tunicamycin (FIGS. 11 and 12). Both of these compounds had been previously demonstrated to induce Parkin expression (Bouman et al., Cell Death and Differentiation, 18: 769-782 (2011). In brief, the validation of the Parkin coincidence reporter assay response by qRT-PCR was carried out as follows. The Parkin coincidence reporter cell line was cultured in 6-well tissue culture plates (200,000 cells/well) and incubated for 16 hours in a tissue culture incubator. Parkin (PARK2) gene expression was induced with 24 hours of treatment of wells with 10 uM Carbonyl cyanide m-chlorophenyl hydrazone or 2 μg/mL Tunicamycin for 12 hours. As a control, a separate sample well was also treated for 24 hours with vehicle alone. At the conclusion of the control or induction treatments, total RNA was isolated (QIAGEN RNA kit) from each sample well and then converted to cDNA with reverse transcriptase (BIO-RAD Kit). TaqMan assays (Life Technologies PARK2, Hs01038325; GAPDH, 4352934E) were used to determine the relative amounts of Parkin mRNA in each sample from the WT PARK2 allele remaining in the cell line. Threshold cycle data generated from qPCR (Applied Biosystems 7900HT instrument) was used to normalize Parkin gene signal to an endogenous control (GAPDH) using the comparative Ct method (Schmittgen et al., Nature Protocols, 3:1101-1108 (2008) (FIG. 11A). In a similar manner, qPCR was performed from the same cDNA samples to quantify the expression of the coincidence reporter cassette mRNA. Additionally, cDNA produced from the parental (pre-genome editing) cell line mRNA was included. In this case, custom qPCR primers were used for the coincidence reporter cassette (Forward 5′-GAATTCTCACGGCTTTCCGC-3′ (SEQ ID NO: 363), and Reverse 5′-GATGCGAGCTGAAGCACAAG-3′ (SEQ ID NO: 364)) and alpha-actin as an endogenous control (Forward 5′-CCCGCCGCCAGCTCACCAT-3′ (SEQ ID NO: 365), and Reverse 5′-CGATGGAGGGGAAGACGGCCC-3′) (SEQ ID NO: 366). A SYBR-Green assay system (Life Technologies) was used to generate the qPCR data. Threshold cycle data from the actin endogenous control pPCR was used to normalize the corresponding coincidence reporter signal in each sample (FIG. 11B). All procedures used standard manufacturer's protocols.

Validation of the Parkin coincidence reporter cell line in 1536-well plates was carried out as follows. The Parkin coincidence reporter cell line seeded at a density of 2000 cell/well into duplicate white, solid bottom, tissue-culture treated (Greiner Bio-One), 1536-well microplates in a total of 5 μL/well of culture medium. After 16 hour incubation in a tissue-culture incubator, a flying reagent dispenser (Beckman-Coulter) was used to add 3 μL of culture medium containing one of the following agents: 1) Vehicle only negative control, 2) CCCP (R2 Positive control), or 3) PTC-124 (R1 Positive control) to blocks of 384 wells on the plate. After reagent dispensing, the final concentration of PRC-124 was 500 nM and CCCP was 10 μM in the respective wells. After a 24 hour incubation in the tissue culture incubator, the volume in each well of both plates was reduced to 2 uL with a microplate aspiration system (BioTek) and then 2 μL of Firefly Luciferase assay reagent (Promega) was added to every well of plate 1 while 2 μL of NanoLuc assay reagent (Promega) was added to every well of plate 2. After a 15 minute incubation at room temperature, the luminescent signal from each well of each plate was measured on a VIEWLUX plate reader (PerkinElmer). The results are shown in FIG. 11C.

Compound library screening in 1536-well plates was carried out as follows. The Parkin coincidence reporter cell line was seeded at a density of 2000 cell/well into white, solid bottom, tissue-culture treated (Greiner Bio-One), 1536-well microplates in a total of 5 μL/well of culture medium. After a 16 hour incubation in a tissue-culture incubator, a compound pin tool (Wako) was used to transfer 20 nL of compound dissolved in DMSO for library plates to the assay plates. Compounds were present in either a 6 or 12-point titration in the library plates. DMSO vehicle, CCCP, and PTC-124 were also added to the designated control well. After a 24 hour incubation in the tissue culture incubator, the volume in each well of both plates was reduced to 2 μL with a microplate aspiration system (BioTek) and then 2 μL of Firefly Luciferase assay reagent (Promega) was added to every well each plate and luminescent signal from each well of each plate was measured on a VIEWLUX plate reader (PerkinElmer). Following the first read, 2 μL of NanoLuc assay reagent (Promega) including a proprietary firefly luciferase inhibitor (to quench the firefly reaction) was added to every well of each plate. After a second 15 minute incubation at room temperature, the NanoLuc signal from each well of each plate was measured on the VIEWLUX. Raw luminescent signal is expressed as a % of the positive control (10 uM CCCP for NanoLuc and 500 nM PTC-124 for FLuc). Examples of the library screening results are shown in FIGS. 12A-12E. As shown in FIGS. 12A and 12B, PTC-124 and Resveratrol are examples of compounds that do not elicit a coincident reporter response and the FLuc signal is obtained through reporter interference. As shown in FIGS. 12C and 12D, Nimodipine and MG-132 are examples of compounds that do not elicit a coincident reporter response and the NLuc signal is obtained through reporter interference. As shown in FIG. 12E, Quercetin is a genuine modulator of endogenous Parkin expression and elicits a coincidence response from both FLuc and NLuc.

### Example 7

This example demonstrates stable, stoichiometric reporter expression.

As shown in FIGS. 13A and 13B, a TRE is either positively (activating) or negatively (repressing) a promoter (P) driving the coincidence reporter. The TRE can occur anywhere on a chromosome in which the coincidence reporter is embedded. Examples of reporter stoichiometry for the constructs shown in FIGS. 13A and 13B are shown in Tables 11A and 11B, respectively. Repeated elements (n=number of copies) encoding either the first reporter (R1)-ribosomal skip sequence (RS) (FIG. 13A) or RS-second reporter (R2) (FIG. 13B) will provide expression of multiple copies of the R1 reporter to a single R2 reporter (FIG. 13A and Table 11A) or multiple copies of the R2 reporter to a single copy of the R1 reporter (FIG. 13B and Table 11B). While n may be any number of copies, examples are shown in Tables 11A and 11B.

TABLE 11A

| N | Ratio of R1:R2 | Reporter stoichiometry |
|---|---|---|
| 1 | 1:1 | equal |
| 2 | 2:1 | 2 R1 for each R2 |
| 3 | 3:1 | 3 R1 for each R2 |

TABLE 11B

| N | Ratio of R1:R2 | Reporter stoichiometry |
|---|---|---|
| 1 | 1:1 | equal |
| 2 | 1:2 | 1 R1 for every 2 R2 |
| 3 | 1:3 | 1 R1 for every 3 R2 |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of screening test compounds for ability to modulate a biological activity of interest, the method comprising:
(a) introducing a nucleic acid into a population of cells, wherein
(i) the nucleic acid comprises a nucleotide sequence encoding two or more reporters including a first reporter and a second reporter that is different from the first reporter,
(ii) the nucleic acid further comprises a nucleotide sequence encoding one or more ribosomal skip sequences, wherein a ribosomal skip sequence is positioned between nucleotide sequences encoding the first and second reporters, and
(iii) the first and second reporters are stoichiometrically co-expressed under control of a transcriptional regulatory element (TRE) and/or promoter that is activated or repressed by modulation of the biological activity of interest;
(b) dividing the cells of (a) into more than one sub-population;
(c) culturing each sub-population of cells with a test compound, wherein each sub-population is cultured with a different test compound;
(d) measuring expression of the first and second reporters in each cultured sub-population of cells; and
(e) identifying at least one test compound modulating the biological activity of interest when both of the first and second reporters are expressed by the sub-population of cells that was cultured with the test compound or when a basal level of expression of both of the first and second reporters is repressed or increased in the sub-population of cells that is cultured with the test compound.

2. The method of claim 1, wherein the biological activity of interest is expression of a target gene.

3. The method of claim 1, wherein the ribosomal skip sequence encodes a Picornavirus 2A peptide or a homolog or variant thereof.

4. The method of claim 1, wherein the TRE is a steroid response element, a heat shock response element, a metal response element, a hormone response element, a cytokine response element, or a serum response element (SRE).

5. The method of claim 1, wherein the TRE is a glucocorticoid receptor element (GRE), an estrogen receptor element (ERE), a cAMP-response element (CRE), a p53 response element, an antioxidant response element (ARE), or a 12-O-tetradecanoylphorbol 13-acetate (TPA) response element.

6. The method of claim 1, wherein the nucleic acid further comprises nucleotide sequences flanking a combination of the nucleotide sequences encoding the two or more reporters and the one or more ribosomal skip sequences, wherein the flanking nucleotide sequences are homologous to a left and right arm of a target site in a genome of the population of cells.

7. A kit for screening test compounds for ability to modulate a biological activity of interest, the kit comprising:
(a) (i) a nucleic acid comprising a nucleotide sequence encoding two or more reporters including a first reporter and a second reporter that is different from the first reporter and one or more ribosomal skip sequences, wherein a ribosomal skip sequence is positioned between the first and second reporters, wherein the first and second reporters are stoichiometrically co-expressed from the nucleotide sequence, and/or (ii) a population of cells comprising the nucleic acid; and
(b) at least one container for holding the nucleic acid or population of cells.

8. The kit of claim 7, comprising the population of cells comprising the nucleic acid, wherein the cells are mammalian cells.

9. The kit of claim 7 or 8, further comprising a cell culture plate.

10. The kit of claim 7, wherein the ribosomal skip sequence encodes a Picornavirus 2A peptide or a homolog or variant thereof.

11. The kit of claim 7, wherein the first and second reporters are co-expressed under control of a transcriptional regulatory element (TRE) and/or promoter that is activated or repressed by modulation of the biological activity of interest.

12. The kit of claim 11, wherein the TRE is a steroid response element, a heat shock response element, a metal response element, a hormone response element, a cytokine response element, or a serum response element (SRE).

13. The kit of claim 11, wherein the TRE is a glucocorticoid receptor element (GRE), an estrogen receptor element (ERE), a cAMP-response element (CRE), a p53 response element, an antioxidant response element (ARE), or a 12-O-tetradecanoylphorbol 13-acetate (TPA) response element.

14. The kit of claim 7, further comprising a first detection reagent that reacts with the first reporter to provide a detectable indicator of the presence or absence of the first reporter and a container for holding the first detection reagent.

15. The kit of claim 14, further comprising a second detection reagent that reacts with the second reporter to provide a detectable indicator of the presence or absence of the second reporter and a container for holding the second detection reagent.

16. A kit for screening test compounds for ability to modulate a biological activity of interest, the kit comprising:
(a) (i) a nucleic acid comprising a nucleotide sequence encoding two or more reporters including a first reporter and a second reporter that is different from the first reporter and one or more ribosomal skip sequences, wherein a ribosomal skip sequence is positioned between the first and second reporters, wherein the first and second reporters are stoichiometrically co-expressed from the nucleotide sequence, and/or (ii) a population of cells comprising the nucleic acid;
(b) at least one container for holding the nucleic acid or population of cells; and
(c) instructions for performing the method of claim 1.

17. The kit of claim 7, wherein the nucleic acid further comprises nucleotide sequences flanking a combination of the nucleotide sequences encoding the two or more reporters and the one or more ribosomal skip sequences, wherein the flanking nucleotide sequences are homologous to a left and right arm of a target site in a genome of the population of cells.

18. A nucleic acid comprising a nucleotide sequence encoding
(i) two or more reporters comprising a first reporter and a second reporter that is different from the first reporter; and
(ii) one or more ribosomal skip sequences, wherein a ribosomal skip sequence is positioned between the first and second reporters,
wherein the first and second reporters are stoichiometrically co-expressed from the nucleotide sequence and the nucleic acid does not comprise a cytomegalovirus-immediate early (CMV-IE) promoter.

19. The nucleic acid of claim 18, wherein the ribosomal skip sequence encodes a Picornavirus 2A peptide or a homolog or variant thereof.

20. The nucleic acid of claim 18 or 19, further comprising a nucleotide sequence comprising a transcriptional regulatory element (TRE) and/or promoter, wherein each of the first and second reporters is operably linked to the TRE and/or promoter.

21. The nucleic acid of claim 20, wherein the TRE is a steroid response element, a heat shock response element, a metal response element, a hormone response element, a cytokine response element, or a serum response element (SRE).

22. The nucleic acid of claim 20, wherein the TRE is a glucocorticoid receptor element (GRE), an estrogen receptor element (ERE), a cAMP-response element (CRE), a p53 response element, an antioxidant response element (ARE), or a 12-O-tetradecanoylphorbol 13-acetate (TPA) response element.

23. The nucleic acid of claim 18, further comprising nucleotide sequences flanking a combination of the nucleotide sequences encoding the two or more reporters and the one or more ribosomal skip sequences, wherein the flanking nucleotide sequences are homologous to a left and right arm of a target site in a genome of the population of cells.

24. A recombinant expression vector comprising the nucleic acid of claim 18.

25. A host cell comprising the recombinant expression vector of claim 24.

26. A population of cells comprising at least one host cell of claim 25.


SEQUENCE LISTING (368 sequences)

SEQ ID NO: 1 - DNA, 57 bp, Artificial Sequence
  Synthetic
gctactaacttcagcctgctgaagcaggctggagacgtggaggagaaccctggacct

SEQ ID NO: 2 - protein, 22 aa, Artificial Sequence
  Synthetic
GSGATNFSLLKQAGDVEENPGP

SEQ ID NO: 3 - DNA, 2658 bp, Artificial Sequence
  Synthetic
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtggaattcggaagcggagctactaacttcagcctgctgaagcaggctggagacgtggaggagaaccctggacctatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagaccttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggacccaggattcttttccaatgctattgttgaaggtgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataa

SEQ ID NO: 4 - protein, 885 aa, Artificial Sequence
  Synthetic
MEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAVEFGSGATNFSLLKQAGDVEENPGPMTSKVYDPEQRKRMITGPQWWARCKQMNVLDSFINYYDSEKHAENAVIFLHGNAASSYLWRHVVPHIEPVARCIIPDLIGMGKSGKSGNGSYRLLDHYKYLTAWFELLNLPKKIIFVGHDWGACLAFHYSYEHQDKIKAIVHAESVVDVIESWDEWPDIEEDIALIKSEEGEKMVLENNFFVETMLPSKIMRKLEPEEFAAYLEPFKEKGEVRRPTLSWPREIPLVKGGKPDVVQIVRNYNAYLRASDDLPKMFIESDPGFFSNAIVEGAKKFPNTEFVKVKGLHFSQEDAPDEMGKYIKSFVERVLKNEQ

SEQ ID NO: 5 - DNA, 2361 bp, Artificial Sequence
  Synthetic
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtggaattcggaagcggagctactaacttcagcctgctgaagcaggctggagacgtggaggagaaccctggacctatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgaattctcacggctttccgcctgaggttgaagagcaagccgccggtacattgcctatgtcctgcgcacaagaaagcggtatggaccggcacccagccgcttgtgcttcagctcgcatcaacgtctaa

SEQ ID NO: 6 - protein, 786 aa, Artificial Sequence
  Synthetic
MEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAVEFGSGATNFSLLKQAGDVEENPGPMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILANSHGFPPEVEEQAAGTLPMSCAQESGMDRHPAACASARINV

SEQ ID NO: 7 - DNA, 2437 bp, Artificial Sequence
  Synthetic
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtggaattcggaagcggagctactaacttcagcctgctgaagcaggctggagacgtggaggagaaccctggacatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 8 - protein, 812 aa, Artificial Sequence
  Synthetic
MEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAVEFGSGATNFSLLKQAGDVEENPGHGEQGRGAVHRGGAHPGRAGRRRKRPQVQRVRRGRGRCHLRQADPEVHLHHRQAARALAHPRDHLHLRRAVLRPLPRPHEAARLLQVRHARRLRPGAHHLLQGRRQLQDPRRGEVRGRHPGEPHRAEGHRLQGGRQHPGAQAGVQLQQPQGLYHRRQAEERHQGELQDPPQHRGRQRAARRPLPAEHPHRRRPRAAARQPLPEHPVRPEQRPQREARSHGPAGVRDRRRDHSRHGRAVQ

SEQ ID NO: 9 - DNA, 1422 bp, Artificial Sequence
  Synthetic
atggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgaattctcacggctttccgcctgaggttgaagagcaagccgccggtacattgcctatgtcctgcgcacaagaaagcggtatggaccggcacccagccgcttgtgcttcagctcgcatcaacgtcgaattcggaagcggagctaccttcagcctgctgaagcaggctggagacgtggaggagaaccctggacctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 10 - protein, 474 aa, Artificial Sequence
  Synthetic
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILANSHGFPPEVEEQAAGTLPMSCAQESGMDRHPAACASARINVEFGSGATFSLLKQAGDVEENPGPMVSKGEELFTGVVPILVELDGDVNGHKFSVSGEGEGDATYGKLTLKFICTTGKLPVPWPTLVTTFTYGVQCFARYPDHMKQHDFFKSAMPEGYVQERTIFFKDDGNYKTRAEVKFEGDTLVNRIELKGIDFKEDGNILGHKLEYNYNSHKVYITADKQKNGIKVNFKTRHNIEDGSVQLADHYQQNTPIGDGPVLLPDNHYLSTQSALSKDPNEKRDHMVLLEFVTAAGITLGMDELYK

SEQ ID NO: 11 - DNA, 1497 bp, Artificial Sequence
  Synthetic
atggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgaattctcacggctttccgcctgaggttgaagagcaagccgccggtacattgcctatgtcctgcgcacaagaaagcggtatggaccggcacccagccgcttgtgcttcagctcgcatcaacgtcgaattcggaagcggagctaccttcagcctgctgaagcaggctggagacgtggaggagaaccctggacctatggacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattgg

SEQ ID NO: 12 - protein, 499 aa, Artificial Sequence
  Synthetic
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILANSHGFPPEVEEQAAGTLPMSCAQESGMDRHPAACASARINVEFGSGATFSLLKQAGDVEENPGPMDPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 13 - DNA, 8 bp, Artificial Sequence
  Synthetic
tgacgtca

SEQ ID NO: 14 - DNA, 53 bp, Artificial Sequence
  Synthetic
tgacgtcagagagcctgacgtcagagagcctgacgtcagagagcctgacgtca

SEQ ID NO: 15 - DNA, 46 bp, Artificial Sequence
  Synthetic
gaagggcggaaagatcgccgtggaattctagagtcggggcggccgg

SEQ ID NO: 16 - DNA, 46 bp, Artificial Sequence
  Synthetic
ccggccgccccgactctagaattccacggcgatctttccgcccttc

SEQ ID NO: 17 - DNA, 111 bp, Artificial Sequence
  Synthetic
cccggcgtcttgaattcggaagcggagctactaacttcagcctgctgaagcaggctggagacgtggaggagaaccctggacctatgacttcgaaagtttatgatccagaac

SEQ ID NO: 18 - DNA, 48 bp, Artificial Sequence
  Synthetic
cccggcgtcttgaattcttattgttcatttttgagaactcgcacaacg

SEQ ID NO: 19 - DNA, 158 bp, Artificial Sequence
  Synthetic
agcttgctcgagatctgcgatctaagagcctgacgtcagagagcctgacgtcagagagcctgacgtcagagagcctgacgtcagaggaattcagacactagagggtatataatggaagctcgacttccagcttggcattccggtactgttggtaaaga

SEQ ID NO: 20 - DNA, 160 bp, Artificial Sequence
  Synthetic
agcttaactttaccaacagtaccggaatgccaagctggaagtcgagcttccattatataccctctagtgtctgaattcctctgacgtcaggctctctgacgtcaggctctctgacgtcaggctctctgacgtcaggctcttagatcgcagatctcgagca

SEQ ID NO: 21 - protein, 19 aa, Artificial Sequence
  Synthetic
AARQMLLLLSGDVETNPGP

SEQ ID NO: 22 - protein, 30 aa, Artificial Sequence
  Synthetic
AFELDLEIESDQIRNKKDLTTEGVEPNPGP

SEQ ID NO: 23 - protein, 30 aa, Artificial Sequence
  Synthetic
AFELHLEIESDQFRNVRDLTTEGVEPNPGP

SEQ ID NO: 24 - protein, 30 aa, Artificial Sequence
  Synthetic
AFELHLEIESDQIRNVRDLTTEGVEPNPGP

SEQ ID NO: 25 - protein, 30 aa, Artificial Sequence
  Synthetic
AFELNLEIESDQIRKKKDLTTEGVEPNPGP

SEQ ID NO: 26 - protein, 30 aa, Artificial Sequence
  Synthetic
AFELNLEIESDQIRNKKDLTTEGVEPNPGP

SEQ ID NO: 27 - protein, 30 aa, Artificial Sequence
  Synthetic
AFELNLEIESDQIRNKKDLTTEGVESNPGP

SEQ ID NO: 28 - protein, 30 aa, Artificial Sequence
  Synthetic
ALPCTCGRAALDARRLLLLASGDVERNPGP

SEQ ID NO: 29 - protein, 30 aa, Artificial Sequence
  Synthetic
ALSCVCGHGNSLLCRLLLFLSGDVEYNPGS

SEQ ID NO: 30 - protein, 30 aa, Artificial Sequence
  Synthetic
ALSCVCGHGNSLLCRLLLFLSGNVEYNPGS

SEQ ID NO: 31 - protein, 30 aa, Artificial Sequence
  Synthetic
ALTTMSLQGPGATNFSLLKQAGDIEENPGP

SEQ ID NO: 32 - protein, 30 aa, Artificial Sequence
  Synthetic
AMTALTFQGPGATNFSLLKQAGDVEENPGP

SEQ ID NO: 33 - protein, 30 aa, Artificial Sequence
  Synthetic
AMTAMAFQGPGATNFSLLKQAGDVEENPGP

SEQ ID NO: 34 - protein, 30 aa, Artificial Sequence
  Synthetic
AMTAMALQGPGATNFSLLKQAGDVEENPGP

SEQ ID NO: 35 - protein, 30 aa, Artificial Sequence
  Synthetic
AMTTISYQGPGATNFSLLKQAGDVEENPGP

SEQ ID NO: 36 - protein, 30 aa, Artificial Sequence
  Synthetic
AMTTLSFQGPGATNFSLLKQAGDVEENPGP

SEQ ID NO: 37 - protein, 30 aa, Artificial Sequence
  Synthetic
AMTTLSLQGPGATNFSLLRQAGDVEENPGP

SEQ ID NO: 38 - protein, 30 aa, Artificial Sequence
  Synthetic
AMTTLSYQGPGATNFSLLKQAGDVEENPGP

SEQ ID NO: 39 - protein, 30 aa, Artificial Sequence
  Synthetic
AMTTLTLQGPGATNFSLLKQAGDVEENPGP

SEQ ID NO: 40 - protein, 30 aa, Artificial Sequence
  Synthetic
AMTTMAFQGPGATNFSLLKQAGDVEENPGP

SEQ ID NO: 41 - protein, 30 aa, Artificial Sequence
  Synthetic
AMTTMLFQGPGAANFSLLRQAGDVEENPGP

SEQ ID NO: 42 - protein, 30 aa, Artificial Sequence
  Synthetic
AMTTMMLQGPGATNFSLLKQAGDVEENPGP

SEQ ID NO: 43 - protein, 30 aa, Artificial Sequence
  Synthetic
AMTTMSFQGPGATNFSLLKQAGDVEENPGP

SEQ ID NO: 44 - protein, 30 aa, Artificial Sequence
  Synthetic
AMTTMSLQGPGATNFSLLKQAGDVEENPGP

SEQ ID NO: 45 - protein, 30 aa, Artificial Sequence
  Synthetic
AMTTMSYQGPGATNFSLLKQAGDVEENPGP

SEQ ID NO: 46 - protein, 30 aa, Artificial Sequence
  Synthetic
AMTTMTFQGRGATNFSLLKQAGDVEENPGP

SEQ ID NO: 47 - protein, 30 aa, Artificial Sequence
  Synthetic
AMTTMTLQGPGATNFSLLKQAGDIEENPGP

SEQ ID NO: 48 - protein, 30 aa, Artificial Sequence
  Synthetic
AMTTMTLQGPGATNFSLLKQAGDVEENPGP

SEQ ID NO: 49 - protein, 30 aa, Artificial Sequence
  Synthetic
AMTVMAFQGPGATNFSLLKQAGDVEENPGP

SEQ ID NO: 50 - protein, 30 aa, Artificial Sequence
  Synthetic
AMTVMTFQGPGATNFSLLKQAGDIEENPGP

SEQ ID NO: 51 - protein, 30 aa, Artificial Sequence
  Synthetic
AMTVMTFQGPGATNFSLLKQAGDVEENPGP

SEQ ID NO: 52 - protein, 30 aa, Artifiical Sequence
AMTVVTYQGPGATNFSLLKQAGDIEENPGP

SEQ ID NO: 53 - protein, 30 aa, Artificial Sequence
  Synthetic
ARELRVSRAERDVAKQLLLIAGDVESNPGP

SEQ ID NO: 54 - protein, 19 aa, Artificial Sequence
  Synthetic
ATNFSLLKQAGDVEENPGP

SEQ ID NO: 55 - protein, 20 aa, Artificial Sequence
  Synthetic
CDAQRQKLLLSGDIEQNPGP

SEQ ID NO: 56 - protein, 30 aa, Artificial Sequence
  Synthetic
CGCFCPLPNVYVPPTHNVLLDGDVESNPGP

SEQ ID NO: 57 - protein, 30 aa, Artificial Sequence
  Synthetic
CGCFCPLPNVYVPPTHNVLLEGDVESNPGP

SEQ ID NO: 58 - protein, 30 aa, Artificial Sequence
  Synthetic
CRRIAYYSNSDCTFRLELLKSGDIQSNPGP

SEQ ID NO: 59 - protein, 30 aa, Artificial Sequence
  Synthetic
DMTRLSFQGPGATNFSLLKQAGDVEENPGP

SEQ ID NO: 60 - protein, 30 aa, Artificial Sequence
  Synthetic
DMTRMSFQGPGATNFSLLKQAGDVEENPGP

SEQ ID NO: 61 - protein, 30 aa, Artificial Sequence
  Synthetic
DMTRMSFQGPGATNFSLLKRAGDVEENPGP

SEQ ID NO: 62 - protein, 30 aa, Artificial Sequence
  Synthetic
DMTRMSLQGPGASNFSLLKQAGDVEENPGP

SEQ ID NO: 63 - protein, 30 aa, Artificial Sequence
  Synthetic
DMTVMTFQGPGATNFSLLKQAGDVEENPGP

SEQ ID NO: 64 - protein, 30 aa, Artificial Sequence
  Synthetic
EATLSTILSEGATNFSLLKLAGDVELNPGP

SEQ ID NO: 65 - protein, 30 aa, Artificial Sequence
  Synthetic
EMTTMSFQGPGATNFSLLKQAGDVEENPGP

SEQ ID NO: 66 - protein, 30 aa, Artificial Sequence
  Synthetic
FFDSIWVYHLANSSWVRDLTRECIESNPGP

SEQ ID NO: 67 - protein, 30 aa, Artificial Sequence
  Synthetic
FFDSVWVYHLANSSWVRDLTRECIESNPGP

SEQ ID NO: 68 - protein, 30 aa, Artificial Sequence
  Synthetic
FGEFFKAVRGYHADYYKQRLIHDVEMNPGP

SEQ ID NO: 69 - protein, 30 aa, Artificial Sequence
  Synthetic
FGEFFKAVRGYHADYYRQRLIHDVETNPGP

SEQ ID NO: 70 - protein, 30 aa, Artificial Sequence
  Synthetic
FGEFFRAVRAYHADYYKQRLIHDVEMNPGP

SEQ ID NO: 71 - protein, 30 aa, Artificial Sequence
  Synthetic
FREFFKAVRGYHADYYKQRLIHDVEMNPGP

SEQ ID NO: 72 - protein, 30 aa, Artificial Sequence
  Synthetic
FSDFFKHVREYHAAYYKQRLMHDVETNPGP

SEQ ID NO: 73 - protein, 30 aa, Artificial Sequence
  Synthetic
FTCTCWRGRALLCRPFLMPLSGDVGQNPEP

SEQ ID NO: 74 - protein, 30 aa, Artificial Sequence
  Synthetic
FTDFFKAVRDYHASYYKQRLQHDIEANPGP

SEQ ID NO: 75 - protein, 30 aa, Artificial Sequence
  Synthetic
FTDFFKAVRDYHASYYKQRLQHDIETPPGP

SEQ ID NO: 76 - protein, 30 aa, Artificial Sequence
  Synthetic
FTDFFKAVRDYHASYYKQRLQHDVETNPGP

SEQ ID NO: 77 - protein, 30 aa, Artificial Sequence
  Synthetic
GAGYPLIVANSKFQIDKILISGDIELNPGP

SEQ ID NO: 78 - protein, 30 aa, Artificial Sequence
  Synthetic
GARIRYYNNSSATFQTILMTCGDVDPNPGP

SEQ ID NO: 79 - protein, 30 aa, Artificial Sequence
  Synthetic
GARISYHPNTTATFQLRLLVSGDVNPNPGP

SEQ ID NO: 80 - protein, 30 aa, Artificial Sequence
  Synthetic
GAVDVVLSQQPYLTELLLVKAGDVELNPGP

SEQ ID NO: 81 - protein, 30 aa, Artificial Sequence
  Synthetic
GIGNPLIVANSKFQIDRILISGDIELNPGP

SEQ ID NO: 82 - protein, 30 aa, Artificial Sequence
  Synthetic
GNGNPLIVANAKFQIDKILISGDVELNPGP

SEQ ID NO: 83 - protein, 30 aa, Artificial Sequence
  Synthetic
GQRTTEQIVTAQGWAPDLTQDGDVESNPGP

SEQ ID NO: 84 - protein, 30 aa, Artificial Sequence
  Synthetic
GQRTTEQIVTAQGWVPDLTVDGDVESNPGP

SEQ ID NO: 85 - protein, 30 aa, Artificial Sequence
  Synthetic
GRRIQYYNNSISTFRSELLRCGDVESNPGP

SEQ ID NO: 86 - protein, 21 aa, Artificial Sequence
  Synthetic
GSGEGRGSLLTCGDVEENPGP

SEQ ID NO: 87 - protein, 23 aa, Artificial Sequence
  Synthetic
GSGQCTNYALLKLAGPVESNPGP

SEQ ID NO: 88 - protein, 25 aa, Artificial Sequence
  Synthetic
GSGVKQTLNFDLLKLAGDVESNPGP

SEQ ID NO: 89 - protein, 30 aa, Artificial Sequence
  Synthetic
GTGYPLIVANSKFQIDKILISGDIELNPGP

SEQ ID NO: 90 - protein, 30 aa, Artificial Sequence
  Synthetic
GVGYPLIVANSKFQIDKILISGDIELNPGP

SEQ ID NO: 91 - protein, 30 aa, Artificial Sequence
  Synthetic
HAANMWDLSTGWFHFFRLLRSGDVESNPGP

SEQ ID NO: 92 - protein, 30 aa, Artificial Sequence
  Synthetic
HKHKIVAPVKQLLNFDLLKLAGDMESNPGP

SEQ ID NO: 93 - protein, 30 aa, Artificial Sequence
  Synthetic
HKQKIIAPAKQLLNFDLLKLAGDVEPNPGP

SEQ ID NO: 94 - protein, 30 aa, Artificial Sequence
  Synthetic
HKQKIIAPAKQLLNFDLLKLAGDVESNPGA

SEQ ID NO: 95 - protein, 30 aa, Artificial Sequence
  Synthetic
HKQKIIAPAKQLLNFDLLKLAGDVESNPGP

SEQ ID NO: 96 - protein, 30 aa, Artificial Sequence
  Synthetic
HKQKIIAPAKQLLNFDLLQLAGDVESNPGP

SEQ ID NO: 97 - protein, 30 aa, Artificial Sequence
  Synthetic
HKQKIIAPAKQSLNFDLLKLAGDVESNPGP

SEQ ID NO: 98 - protein, 30 aa, Artificial Sequence
  Synthetic
HKQKIIAPARQLLNFDLLKLAGDVESNPGP

SEQ ID NO: 99 - protein, 30 aa, Artificial Sequence
  Synthetic
HKQKIIAPEKQLLNFDLLKLAGDVESNPGP

SEQ ID NO: 100 - protein, 30 aa, Artificial Sequence
  Synthetic
HKQKIIAPGKQLLNFDLLKLAGDVESNPGP

SEQ ID NO: 101 - protein, 30 aa, Artificial Sequence
  Synthetic
HKQKIIAPGKQLLNFDLLKLAGDVESNPRP

SEQ ID NO: 102 - protein, 30 aa, Artificial Sequence
  Synthetic
HKQKIIAPSKQLLNFDLLKLAGDVESNPGP

SEQ ID NO: 103 - protein, 30 aa, Artificial Sequence
  Synthetic
HKQKIIAPTKQLLNFDLLKLAGDVESNPGP

SEQ ID NO: 104 - protein, 30 aa, Artificial Sequence
  Synthetic
HKQKIIAPVKQLLNFDLLKLAGDVESNPGP

SEQ ID NO: 105 - protein, 30 aa, Artificial Sequence
  Synthetic
HKQKIITPVKQLLNFDLLKLAGDVESNPGP

SEQ ID NO: 106 - protein, 30 aa, Artificial Sequence
  Synthetic
HKQKIVAPAKQLLNFDLLKLAGDVESNPGP

SEQ ID NO: 107 - protein, 30 aa, Artificial Sequence
  Synthetic
HKQKIVAPAKQSLNFDLLRLAGDVESNPGP

SEQ ID NO: 108 - protein, 30 aa, Artificial Sequence
  Synthetic
HKQKIVAPAKQTLNFDLLKLAGDVESNPGP

SEQ ID NO: 109 - protein, 30 aa, Artificial Sequence
  Synthetic
HKQKIVAPTKQLLNFDLLKLAGDVESNPGP

SEQ ID NO: 110 - protein, 30 aa, Artificial Sequence
  Synthetic
HKQKIVAPVKQLLNFDLLKLAGDVEPNPGP

SEQ ID NO: 111 - protein, 30 aa, Artificial Sequence
  Synthetic
HKQKIVAPVKQLLNFDLLKLAGDVESNLGP

SEQ ID NO: 112 - protein, 30 aa, Artificial Sequence
  Synthetic
HKQKIVAPVKQLLNFDLLKLAGDVESNPGA

SEQ ID NO: 113 - protein, 30 aa, Artificial Sequence
  Synthetic
HKQKIVAPVKQLLNFDLLKLAGDVESNPGP

SEQ ID NO: 114 - protein, 30 aa, Artificial Sequence
  Synthetic
HKQKIVAPVKQLLNFDLLKLAGDVESNQGA

SEQ ID NO: 115 - protein, 30 aa, Artificial Sequence
  Synthetic
HKQKIVAPVKQLLNFELLKLAGDVESNPGP

SEQ ID NO: 116 - protein, 30 aa, Artificial Sequence
  Synthetic
HKQKIVAPVKQLLNFNLLKLAGDVESNPGP

SEQ ID NO: 117 - protein, 30 aa, Artificial Sequence
  Synthetic
HKQKIVAPVKQLLSFDLLKLAGDVESNPGP

SEQ ID NO: 118 - protein, 30 aa, Artificial Sequence
  Synthetic
HKQKIVAPVKQTLNFDLLKLAGDVESNPGP

SEQ ID NO: 119 - protein, 30 aa, Artificial Sequence
  Synthetic
HKQPLIAPAKQLLNFDLLKLAGDVESNPGP

SEQ ID NO: 120 - protein, 30 aa, Artificial Sequence
  Synthetic
HKQPLIAPAKQLSNFDLLKLAGDVESNPGP

SEQ ID NO: 121 - protein, 30 aa, Artificial Sequence
  Synthetic
HKQPLIAPEKQLLNFDLLKLAGDVESNPGP

SEQ ID NO: 122 - protein, 30 aa, Artificial Sequence
  Synthetic
HKQPLVAPAKQLLNFDLLKLAGDVESNPGP

SEQ ID NO: 123 - protein, 30 aa, Artificial Sequence
  Synthetic
HKQRIIAPAKQLLNFDLLKLAGDVESNLGP

SEQ ID NO: 124 - protein, 30 aa, Artificial Sequence
  Synthetic
HKQRIIAPAKQLLNFDLLKLAGDVESNPGA

SEQ ID NO: 125 - protein, 30 aa, Artificial Sequence
  Synthetic
HKQRIIAPAKQLLNFDLLKLAGDVESNPGP

SEQ ID NO: 126 - protein, 30 aa, Artificial Sequence
  Synthetic
HKQRIIAPAKQLLNFDLLQLAGDVESNPGP

SEQ ID NO: 127 - protein, 30 aa, Artificial Sequence
  Synthetic
HKQRIVAPAKQLLNFDLLKLAGDVESNPGP

SEQ ID NO: 128 - protein, 30 aa, Artificial Sequence
  Synthetic
HKQSIIAPAKQLLNFDLLKLAGDVESNPGP

SEQ ID NO: 129 - protein, 30 aa, Artificial Sequence
  Synthetic
HKTALVKPAKQLCNFDLLKLAGDVESNPGP

SEQ ID NO: 130 - protein, 20 aa, Artificial Sequence
  Synthetic
HYAGYFADLLIHDIETNPGP

SEQ ID NO: 131 - protein, 30 aa, Artificial Sequence
  Synthetic
IFGLYRIFSTHYAGYFSDLLIHDIETNPGP

SEQ ID NO: 132 - protein, 30 aa, Artificial Sequence
  Synthetic
IGFLNKLYKCGTWESVLNLLAGDIELNPGP

SEQ ID NO: 133 - protein, 30 aa, Artificial Sequence
  Synthetic
IGFLNKLYRCGDWDSILLLLSGDIEENPGP

SEQ ID NO: 134 - protein, 30 aa, Artificial Sequence
  Synthetic
IHANDYQMAVFKSNYDLLKLCGDVESNPGP

SEQ ID NO: 135 - protein, 30 aa, Artificial Sequence
  Synthetic
IIARPYIRESSNVSRLKLLLSGDIETNPGP

SEQ ID NO: 136 - protein, 30 aa, Artificial Sequence
  Synthetic
ILPCACGRAALDARRLLLLASGDVGRNPGP

SEQ ID NO: 137 - protein, 30 aa, Artificial Sequence
  Synthetic
ILPCACGRATLDARRLLVLISGDVERNPGA

SEQ ID NO: 138 - protein, 30 aa, Artificial Sequence
  Synthetic
ILPCACGRATLGARRLLLLISGDVERNPGP

SEQ ID NO: 139 - protein, 30 aa, Artificial Sequence
  Synthetic
ILPCACGRAVSDALRLLLLISGDVECNPGP

SEQ ID NO: 140 - protein, 30 aa, Artificial Sequence
  Synthetic
ILPCLCVHAASDARWLLLLISGDVERRPCP

SEQ ID NO: 141 - protein, 30 aa, Artificial Sequence
  Synthetic
ILPCMCGRATLDARRLLLLVSEDIERNPGP

SEQ ID NO: 142 - protein, 30 aa, Artificial Sequence
  Synthetic
ILPCTCERATLDARRLLLLISGDVERNPGP

SEQ ID NO: 143 - protein, 30 aa, Artificial Sequence
  Synthetic
ILPCTCGCATLDARRILLLVSGDVERNPGP

SEQ ID NO: 144 - protein, 30 aa, Artificial Sequence
  Synthetic
ILPCTCGHAALDARRRLLLISGDVERNPGA

SEQ ID NO: 145 - protein, 30 aa, Artificial Sequence
  Synthetic
ILPCTCGHAALDARRRPLLVGRDVKRNPGP

SEQ ID NO: 146 - protein, 30 aa, Artificial Sequence
  Synthetic
ILPCTCGRAALDAQWRLLLIFVDAERNPGP

SEQ ID NO: 147 - protein, 30 aa, Artificial Sequence
  Synthetic
ILPCTCGRAALDARRLLLLISGNVECNPGP

SEQ ID NO: 148 - protein, 30 aa, Artificial Sequence
  Synthetic
ILPCTCGRAALDVRRHLLLIIGDVERNPGP

SEQ ID NO: 149 - protein, 30 aa, Artificial Sequence
  Synthetic
ILPCTCGRAASDVRRLLLLIGGDAERNPGP

SEQ ID NO: 150 - protein, 30 aa, Artificial Sequence
  Synthetic
ILPCTCGRAMLDARRLLLLISVDVERNPGP

SEQ ID NO: 151 - protein, 30 aa, Artificial Sequence
  Synthetic
ILPCTCGRATLDAPRILLLVSGDVERNPGP

SEQ ID NO: 152 - protein, 30 aa, Artificial Sequence
  Synthetic
ILPCTCGRATLDAQRILLLVSGDVERNPGP

SEQ ID NO: 153 - protein, 30 aa, Artificial Sequence
  Synthetic
ILPCTCGRATLDARRFLLPVRGDVGRNPGP

SEQ ID NO: 154 - protein, 30 aa, Artificial Sequence
  Synthetic
ILPCTCGRATLDARRILLLVSGDIERNPGP

SEQ ID NO: 155 - protein, 30 aa, Artificial Sequence
  Synthetic
ILPCTCGRATLDARRILLLVSGDVERNPGP

SEQ ID NO: 156 - protein, 30 aa, Artificial Sequence
  Synthetic
ILPCTCGRATLDARRLLLLASGDVERNPGP

SEQ ID NO: 157 - protein, 30 aa, Artificial Sequence
  Synthetic
ILPCTCGRATLDARRLLLLISGAVERNPGP

SEQ ID NO: 158 - protein, 30 aa, Artificial Sequence
  Synthetic
ILPCTCGRATLDARRLLLLISGDVERNPGP

SEQ ID NO: 159 - protein, 30 aa, Artificial Sequence
  Synthetic
ILPCTCGRATLDARRLLLLISGDVERNPVP

SEQ ID NO: 160 - protein, 30 aa, Artificial Sequence
  Synthetic
ILPCTCGRATLDARRTLLLISGDVERNPGP

SEQ ID NO: 161 - protein, 30 aa, Artificial Sequence
  Synthetic
ILPCTCGRATLDVLRLLLLVSGDVERNSGP

SEQ ID NO: 162 - protein, 30 aa, Artificial Sequence
  Synthetic
ILPCTCGRATLGARRLLLLISVDVERNPGP

SEQ ID NO: 163 - protein, 30 aa, Artificial Sequence
  Synthetic
ILPCTCGRAVSDARRLLLLISGDVGRNPGP

SEQ ID NO: 164 - protein, 30 aa, Artificial Sequence
  Synthetic
ILPCTCGRTTLDARRILLLVSGDIERNPGP

SEQ ID NO: 165 - protein, 30 aa, Artificial Sequence
  Synthetic
ILPCTCICPTLEARRLLVLVSGGIERNPRP

SEQ ID NO: 166 - protein, 30 aa, Artificial Sequence
  Synthetic
ILPCTRGRAMLSARWLLLLISGGVERKPGP

SEQ ID NO: 167 - protein, 30 aa, Artificial Sequence
  Synthetic
ILPCTRGRATLDARRLLLLVSGGVERNPGP

SEQ ID NO: 168 - protein, 30 aa, Artificial Sequence
  Synthetic
ILPCTRGRATLDARRPLLLISGVVERNPGP

SEQ ID NO: 169 - protein, 30 aa, Artificial Sequence
  Synthetic
ILPFTCGRAALDAWRLLLLIGGGVGRNPGP

SEQ ID NO: 170 - protein, 30 aa, Artificial Sequence
  Synthetic
ILPFTCGRAGLDTRRLLLLISGGVGRNPGP

SEQ ID NO: 171 - protein, 30 aa, Artificial Sequence
  Synthetic
ILPFTCGRAGLDTRRLPLLISGGVGRNPGP

SEQ ID NO: 172 - protein, 30 aa, Artificial Sequence
  Synthetic
ILPRTCGRATLDAQRILLLVSGDVKRNPGP

SEQ ID NO: 173 - protein, 30 aa, Artificial Sequence
  Synthetic
ILPRTCGRATLDARRLLLLIDGDVERIPGP

SEQ ID NO: 174 - protein, 30 aa, Artificial Sequence
  Synthetic
ILPRTCGRATLDARRRPLLVGRGVERNPGP

SEQ ID NO: 175 - protein, 30 aa, Artificial Sequence
  Synthetic
ILPRTCGSATLDARRRLLLISGDVERMPGP

SEQ ID NO: 176 - protein, 30 aa, Artificial Sequence
  Synthetic
ILPRTCGSATLDARRRLLLISGDVERTPGP

SEQ ID NO: 177 - protein, 30 aa, Artificial Sequence
  Synthetic
ILPYTCECATLDALRLLLLTCGDVERNPGP

SEQ ID NO: 178 - protein, 30 aa, Artificial Sequence
  Synthetic
IVPCTCGRTTLDARRILLLVSGDIERNPGP

SEQ ID NO: 179 - protein, 30 aa, Artificial Sequence
  Synthetic
KAYRMCKEFVRESDNQELLKCGDVESNPGP

SEQ ID NO: 180 - protein, 30 aa, Artificial Sequence
  Synthetic
KLPCTCRRAALDARRLLLLINGGVERNPGP

SEQ ID NO: 181 - protein, 30 aa, Artificial Sequence
  Synthetic
KQTEDHCTNKQLLNFDLLKLAGDVESNPGP

SEQ ID NO: 182 - protein, 30 aa, Artificial Sequence
  Synthetic
KRRIPYNPNSTASFQLELLHAGDVHPNPGP

SEQ ID NO: 183 - protein, 30 aa, Artificial Sequence
  Synthetic
KSCISYYSNSTACFNIEIMCCGDVKSNPGP

SEQ ID NO: 184 - protein, 30 aa, Artificial Sequence
  Synthetic
KTRIPYSVNSNASFQLELLHAGDVHPNPGP

SEQ ID NO: 185 - protein, 30 aa, Artificial Sequence
  Synthetic
LCPLDFRSTSLSHLTILLLLSGQVETNPDP

SEQ ID NO: 186 - protein, 30 aa, Artificial Sequence
  Synthetic
LCPLDFRSTSLSHLTILLLLSGQVETNPGP

SEQ ID NO: 187 - protein, 30 aa, Artificial Sequence
  Synthetic
LEKLVERRTRVCHVGCALFISVDVELNPGP

SEQ ID NO: 188 - protein, 30 aa, Artificial Sequence
  Synthetic
LEMKESNSGYVVGGRGSLLTCGDVESNPGP

SEQ ID NO: 189 - protein, 30 aa, Artificial Sequence
  Synthetic
LHPAILCSASLCFRPYLLLMAGDVEPNPGP

SEQ ID NO: 190 - protein, 30 aa, Artificial Sequence
  Synthetic
LLACTCGRAALDVRRRLLLISGTVKRDPGP

SEQ ID NO: 191 - protein, 30 aa, Artificial Sequence
  Synthetic
LLACTCGRAALDVRRRLLLISGTVKRNPGP

SEQ ID NO: 192 - protein, 30 aa, Artificial Sequence
  Synthetic
LLACTCGRAALDVRRRLLRITGTVKRNPGP

SEQ ID NO: 193 - protein, 30 aa, Artificial Sequence
  Synthetic
LLACTFGRAALDERRRLLRISGTVKRDPGP

SEQ ID NO: 194 - protein, 19 aa, Artificial Sequence
  Synthetic
LLNFDLLKLAGDVESNPGP

SEQ ID NO: 195 - protein, 30 aa, Artificial Sequence
  Synthetic
LLPCTCGRAALDARRLLLLIIGGVERKPGP

SEQ ID NO: 196 - protein, 30 aa, Artificial Sequence
  Synthetic
LLPCTCGRATLDARRLLLLINGDVERNPGP

SEQ ID NO: 197 - protein, 30 aa, Artificial Sequence
  Synthetic
LLPCTCGRATLDAWRLLLLICGGVGRNPGP

SEQ ID NO: 198 - protein, 30 aa, Artificial Sequence
  Synthetic
LLSTCGSALPKALRPPLLLLSRDEDHNPGP

SEQ ID NO: 199 - protein, 30 aa, Artificial Sequence
  Synthetic
LRHPNRQCALQEALRQKLLLCGDVEANPGP

SEQ ID NO: 200 - protein, 30 aa, Artificial Sequence
  Synthetic
LRHPNRQCALQEALRQKLPLCGDVEANPGP

SEQ ID NO: 201 - protein, 30 aa, Artificial Sequence
  Synthetic
LRHPNRQYALQEALRQKFLLCGDVESNPGP

SEQ ID NO: 202 - protein, 30 aa, Artificial Sequence
  Synthetic
LRLTGEIVKQGATNFELLQQAGDVETNPGP

SEQ ID NO: 203 - protein, 30 aa, Artificial Sequence
  Synthetic
LVSSNDECRAFLRKRTQLLMSGDVESNPGP

SEQ ID NO: 204 - protein, 30 aa, Artificial Sequence
  Synthetic
MAASDGLAPRKYLSYRKIQLSGDVETNPGP

SEQ ID NO: 205 - protein, 30 aa, Artificial Sequence
  Synthetic
MHPCTRGRAVLDARRLPLLISGDVERNPGP

SEQ ID NO: 206 - protein, 30 aa, Artificial Sequence
  Synthetic
MLLCTRGCAMLDARRLLLPVRGDVERNPGT

SEQ ID NO: 207 - protein, 30 aa, Artificial Sequence
  Synthetic
MLLCTRGRAMLRARWLLLLISGDVERDPGP

SEQ ID NO: 208 - protein, 30 aa, Artificial Sequence
  Synthetic
MLLCTSGRAMLRARWLLLLISGDVERDSGP

SEQ ID NO: 209 - protein, 30 aa, Artificial Sequence
  Synthetic
MLPCACGRATLDARRLTLLVSGDVERDPGP

SEQ ID NO: 210 - protein, 30 aa, Artificial Sequence
  Synthetic
MLPCTCGRATLDARRLLLLIIGDVERDPGP

SEQ ID NO: 211 - protein, 30 aa, Artificial Sequence
  Synthetic
MLPCTCGRATLDARRLLLLISGDVERNPGP

SEQ ID NO: 212 - protein, 30 aa, Artificial Sequence
  Synthetic
MTAFDFQQAVFRSNYDLLKLCGDVESNPGP

SEQ ID NO: 213 - protein, 30 aa, Artificial Sequence
  Synthetic
NMARMSFQGPGATNFSLLKQAGDVEENPGP

SEQ ID NO: 214 - protein, 30 aa, Artificial Sequence
  Synthetic
NSDDEEPEYPRGDPIEDLTDDGDIEKNPGP

SEQ ID NO: 215 - protein, 30 aa, Artificial Sequence
  Synthetic
NSSCVLNIRSTSHLAILLLLSGQVEPNPGP

SEQ ID NO: 216 - protein, 30 aa, Artificial Sequence
  Synthetic
NSTPAAMFVCAFILISVLLLSGDVEINPGP

SEQ ID NO: 217 - protein, 30 aa, Artificial Sequence
  Synthetic
NSTPAAMFVCVFILISVLLLSGDVEISPGP

SEQ ID NO: 218 - protein, 30 aa, Artificial Sequence
  Synthetic
NTSLRVLACCVRRAAAPAVYQRDVERKPGP

SEQ ID NO: 219 - protein, 30 aa, Artificial Sequence
  Synthetic
PELNGDQRATLSAWTRDLTKDGDVESNPGP

SEQ ID NO: 220 - protein, 30 aa, Artificial Sequence
  Synthetic
PPRPLSTSIRSRAAYLRQKLMHDIETNPGP

SEQ ID NO: 221 - protein, 30 aa, Artificial Sequence
  Synthetic
PQQDLQGFCLLYLLMILLMRSGDVETNPGP

SEQ ID NO: 222 - protein, 30 aa, Artificial Sequence
  Synthetic
PSIGNVARTLTRAEIEDELIRAGIESNPGP

SEQ ID NO: 223 - protein, 20 aa, Artificial Sequence
  Synthetic
QCTNYALLKLAGDVESNPGP

SEQ ID NO: 224 - protein, 30 aa, Artificial Sequence
  Synthetic
QDLDVKEADKPHITQSLILKAGDVESNPGP

SEQ ID NO: 225 - protein, 30 aa, Artificial Sequence
  Synthetic
QGIGKKNPKQEAARQMLLLLSGDVETNPGP

SEQ ID NO: 226 - protein, 30 aa, Artificial Sequence
  Synthetic
QNLDFNLYLLMILLMILLMRSGDVETNPGP

SEQ ID NO: 227 - protein, 30 aa, Artificial Sequence
  Synthetic
QPYTYCLRALCDAQRQKLLLIGDIEQNPGP

SEQ ID NO: 228 - protein, 30 aa, Artificial Sequence
  Synthetic
QRYTYRLRAVCDAQRQKLLLSGDIEQNPGP

SEQ ID NO: 229 - protein, 20 aa, Artificial Sequence
  Synthetic
RAEGRGSLLTCGDVEENPGP

SEQ ID NO: 230 - protein, 30 aa, Artificial Sequence
  Synthetic
RAWCPSMLPFRSYKQKMLMQSGDIETNPGP

SEQ ID NO: 231 - protein, 30 aa, Artificial Sequence
  Synthetic
RDVRYIEKPFDKEEHTDILLSGDVEENPGP

SEQ ID NO: 232 - protein, 30 aa, Artificial Sequence
  Synthetic
RFDAPIGVAKQLLNFDLLKLAGDVESNPGP

SEQ ID NO: 233 - protein, 30 aa, Artificial Sequence
  Synthetic
RFDAPIGVEKQLCNCDLLKLAGDVESNPGP

SEQ ID NO: 234 - protein, 30 aa, Artificial Sequence
  Synthetic
RFDAPIGVEKQLFNFDLLKLAGDVESNPGP

SEQ ID NO: 235 - protein, 30 aa, Artificial Sequence
  Synthetic
RFDAPIGVEKQLLNFDLLKLAGDVESNPGP

SEQ ID NO: 236 - protein, 30 aa, Artificial Sequence
  Synthetic
RFDSPIGVKKQLCNFDLLKLAGDVESNPGP

SEQ ID NO: 237 - protein, 30 aa, Artificial Sequence
  Synthetic
RGPRPQNLGVRAEGRGSLLTCGDVEENPGP

SEQ ID NO: 238 - protein, 30 aa, Artificial Sequence
  Synthetic
RHKEDCAPVKQLLNFDLLKLAGDVESNPGP

SEQ ID NO: 239 - protein, 30 aa, Artificial Sequence
  Synthetic
RHKFPTNINKQCTNYALLKLAGDVESNPGP

SEQ ID NO: 240 - protein, 30 aa, Artificial Sequence
  Synthetic
RHKFPTNINKQCTNYSLLKLAGDVESNPGP

SEQ ID NO: 241 - protein, 30 aa, Artificial Sequence
  Synthetic
RHNEDCAPVKQLLNFDLLKLAGDVESNPGP

SEQ ID NO: 242 - protein, 30 aa, Artificial Sequence
  Synthetic
RHNEDCATLEQLLNFDLLKLAGDVESNPGP

SEQ ID NO: 243 - protein, 30 aa, Artificial Sequence
  Synthetic
RKQEIIAPAKQMMNFDLLKLAGDVESNPGP

SEQ ID NO: 244 - protein, 30 aa, Artificial Sequence
  Synthetic
RKQEIIAPEKQALNFDLLELAGDVESNPGP

SEQ ID NO: 245 - protein, 30 aa, Artificial Sequence
  Synthetic
RKQEIIAPEKQALNFDLLKLAGDVESNPGP

SEQ ID NO: 246 - protein, 30 aa, Artificial Sequence
  Synthetic
RKQEIIAPEKQDLNLDLLKLAGDVESNPGP

SEQ ID NO: 247 - protein, 30 aa, Artificial Sequence
  Synthetic
RKQEIIAPEKQLLNFDLLKLAGDVESNPGP

SEQ ID NO: 248 - protein, 30 aa, Artificial Sequence
  Synthetic
RKQEIIAPEKQMMNFDLLKLAGDVEPNPGP

SEQ ID NO: 249 - protein, 30 aa, Artificial Sequence
  Synthetic
RKQEIIAPEKQMMNFDLLKLAGDVESNPGA

SEQ ID NO: 250 - protein, 30 aa, Artificial Sequence
  Synthetic
RKQEIIAPEKQMMNFDLLKLAGDVESNPGP

SEQ ID NO: 251 - protein, 30 aa, Artificial Sequence
  Synthetic
RKQEIIAPEKQMMNFELLKLAGDVESNPGP

SEQ ID NO: 252 - protein, 30 aa, Artificial Sequence
  Synthetic
RKQEIIAPEKQTLNFDLLKLAGDVESNPGP

SEQ ID NO: 253 - protein, 30 aa, Artificial Sequence
  Synthetic
RKQEIIAPEKQVLNFDLLKLAGDVESNLGP

SEQ ID NO: 254 - protein, 30 aa, Artificial Sequence
  Synthetic
RKQEIIAPEKQVLNFDLLKLAGDVESNPGP

SEQ ID NO: 255 - protein, 30 aa, Artificial Sequence
  Synthetic
RKQEIIAPEKQVLNFDLLKLSGDVESNPGP

SEQ ID NO: 256 - protein, 30 aa, Artificial Sequence
  Synthetic
RKQEIIAPEKQVLNLDLLKLAGDVESNPGP

SEQ ID NO: 257 - protein, 30 aa, Artificial Sequence
  Synthetic
RKQEIIAPKKQVLNFDLLKLAGDVESNPGP

SEQ ID NO: 258 - protein, 30 aa, Artificial Sequence
  Synthetic
RKQKIIAPEKQLLNFDLLKLAGDVESNPGP

SEQ ID NO: 259 - protein, 30 aa, Artificial Sequence
  Synthetic
RKQKIIAPEKQMMNFDLLKLAGDVESNPGP

SEQ ID NO: 260 - protein, 30 aa, Artificial Sequence
  Synthetic
RKQKIIAPEKQTLNFDLLKLAGDVESNPGP

SEQ ID NO: 261 - protein, 30 aa, Artificial Sequence
  Synthetic
RKQKIIAPEKQVLNFDLLKLAGDVESNPGP

SEQ ID NO: 262 - protein, 30 aa, Artificial Sequence
  Synthetic
RKQKIIAPGKQALNFDLLKLAGDVELNPGP

SEQ ID NO: 263 - protein, 30 aa, Artificial Sequence
  Synthetic
RKQKIIAPGKQVMNFDLLKLAGDVELNPGP

SEQ ID NO: 264 - protein, 30 aa, Artificial Sequence
  Synthetic
RKQPLVAPAKQLLNFDLLKLAGDVESNPGP

SEQ ID NO: 265 - protein, 30 aa, Artificial Sequence
  Synthetic
RKQPLVAPAKQLLNFGLLKLAGDVESNPGP

SEQ ID NO: 266 - protein, 30 aa, Artificial Sequence
  Synthetic
RKQQLVAPAKQLLNFDLLKLAGDVESNPGP

SEQ ID NO: 267 - protein, 30 aa, Artificial Sequence
  Synthetic
RRLPESAQLPQGAGRGSLVTCGDVEENPGP

SEQ ID NO: 268 - protein, 30 aa, Artificial Sequence
  Synthetic
RSLGTCKRAISSIIRTKMLVSGDVEENPGP

SEQ ID NO: 269 - protein, 30 aa, Artificial Sequence
  Synthetic
RSLGTCQRAISSIIRTKMLLSGDVEENPGP

SEQ ID NO: 270 - protein, 30 aa, Artificial Sequence
  Synthetic
RTAFDFQQDVFRSNYDLLKLCGDIESNPGP

SEQ ID NO: 271 - protein, 30 aa, Artificial Sequence
  Synthetic
SFLNTSLRVRVRHVGCALFISVDVELNPGP

SEQ ID NO: 272 - protein, 30 aa, Artificial Sequence
  Synthetic
SGCFCPLPNVYVPPIHNVLLDGDVESNPGP

SEQ ID NO: 273 - protein, 30 aa, Artificial Sequence
  Synthetic
SGCFCPLPNVYVPPTHNVLLDGDVESNPGP

SEQ ID NO: 274 - protein, 30 aa, Artificial Sequence
  Synthetic
SGCFCPLPNVYVPPTHNVLLDGDVESNPRP

SEQ ID NO: 275 - protein, 30 aa, Artificial Sequence
  Synthetic
SKTDLISGQFPPLSELLLLKSGDVELNPGP

SEQ ID NO: 276 - protein, 30 aa, Artificial Sequence
  Synthetic
SKTDLISGQIPHLSELLLMKSGDVELNPGP

SEQ ID NO: 277 - protein, 30 aa, Artificial Sequence
  Synthetic
SKTDLISGQIPPLSELLLLKSGDVELNPGP

SEQ ID NO: 278 - protein, 30 aa, Artificial Sequence
  Synthetic
SKTDLISGQIPPLSELLLMKSGDVELNPGP

SEQ ID NO: 279 - protein, 30 aa, Artificial Sequence
  Synthetic
SKTDLISGQIPPLSKLLLLKSGDVELNPGP

SEQ ID NO: 280 - protein, 30 aa, Artificial Sequence
  Synthetic
SKTDLISGQIPSLSELLLLKSGDVELNPGP

SEQ ID NO: 281 - protein, 30 aa, Artificial Sequence
  Synthetic
SKTELMSGQIPPLSELLLLKSGDVELNPGP

SEQ ID NO: 282 - protein, 30 aa, Artificial Sequence
  Synthetic
SQNIDVLSQQPYLTELLLVKAGDVELNPGP

SEQ ID NO: 283 - protein, 30 aa, Artificial Sequence
  Synthetic
SQRDLSCSQPRTIILGLIMCAGDVQPNPGP

SEQ ID NO: 284 - protein, 30 aa, Artificial Sequence
  Synthetic
SQVRWSNGAEKKVQRLLLLSGGDVERNPGP

SEQ ID NO: 285 - protein, 30 aa, Artificial Sequence
  Synthetic
SRPILYYSNTTASFQLSTLLSGDIEPNPGP

SEQ ID NO: 286 - protein, 30 aa, Artificial Sequence
  Synthetic
SSLNTSLRVRVCHVGCALFISVDVELNPGP

SEQ ID NO: 287 - protein, 30 aa, Artificial Sequence
  Synthetic
SSLSTSLRVRLCHVGCALFISVDVELNPGP

SEQ ID NO: 288 - protein, 30 aa, Artificial Sequence
  Synthetic
SSLSTSLRVRVCHVGCALFISVDVELNPGP

SEQ ID NO: 289 - protein, 30 aa, Artificial Sequence
  Synthetic
TGFLNKLYHCGSWTDILLLLSGDVETNPGP

SEQ ID NO: 290 - protein, 30 aa, Artificial Sequence
  Synthetic
TGFLNKLYHCGSWTDILLLWSGDVETNPGP

SEQ ID NO: 291 - protein, 30 aa, Artificial Sequence
  Synthetic
TLFCTCGSALPKALRPLLLLSRVEDHNPGP

SEQ ID NO: 292 - protein, 30 aa, Artificial Sequence
  Synthetic
TLMGNIMTLAGSGGRGSLLTAGDVEKNPGP

SEQ ID NO: 293 - protein, 30 aa, Artificial Sequence
  Synthetic
TLPFARWHIALDMRRPLLLISGDVDSKPGP

SEQ ID NO: 294 - protein, 30 aa, Artificial Sequence
  Synthetic
TLSCTCGSALPKALGPLLLLSRVEDHNPGP

SEQ ID NO: 295 - protein, 30 aa, Artificial Sequence
  Synthetic
TLSCTCGSALPKALRPLLLPSRDVERNPGP

SEQ ID NO: 296 - protein, 30 aa, Artificial Sequence
  Synthetic
TMTTLSFQGPGATNFSLLKQAGDVEENPGP

SEQ ID NO: 297 - protein, 30 aa, Artificial Sequence
  Synthetic
TMTTMSFQGPGASSFSLLKQAGDVEENPGP

SEQ ID NO: 298 - protein, 30 aa, Artificial Sequence
  Synthetic
TMTTMSLQGPGATNFSLLKQAGDVEENPGP

SEQ ID NO: 299 - protein, 30 aa, Artificial Sequence
  Synthetic
TMTVVSFQGPGATNFSLLKQAGDVEENPGP

SEQ ID NO: 300 - protein, 30 aa, Artificial Sequence
  Synthetic
TQTEDHCTSKQLLNFDLLKLAGDVESNPGP

SEQ ID NO: 301 - protein, 30 aa, Artificial Sequence
  Synthetic
TQTGDHCTSKQLLNFDLLKLAGDVESNPGP

SEQ ID NO: 302 - protein, 20 aa, Artificial Sequence
  Synthetic
TRAEIEDELIRAGIESNPGP

SEQ ID NO: 303 - protein, 30 aa, Artificial Sequence
  Synthetic
TRGGLQRQNIIGGGQRDLTQDGDIESNPGP

SEQ ID NO: 304 - protein, 30 aa, Artificial Sequence
  Synthetic
TRGGLRRQNIIGGGQKDLTQDGDIESNPGP

SEQ ID NO: 305 - protein, 30 aa, Artificial Sequence
  Synthetic
TTCQCKALSVMYLTLLLLTNASDIELNPGP

SEQ ID NO: 306 - protein, 30 aa, Artificial Sequence
  Synthetic
TTDDPVVQESTCLPEMILVKAGDVEQNPGP

SEQ ID NO: 307 - protein, 30 aa, Artificial Sequence
  Synthetic
TVPPNRQCALQEALRKKLLLCGDVESNPWN

SEQ ID NO: 308 - protein, 30 aa, Artificial Sequence
  Synthetic
VADWENLLSQGATNFDLLKLAGDVESNPGP

SEQ ID NO: 309 - protein, 30 aa, Artificial Sequence
  Synthetic
VFGLYGIFNAHYAGYFADLLIHDIETNPGP

SEQ ID NO: 310 - protein, 30 aa, Artificial Sequence
  Synthetic
VFGLYHVFETHYAGYFSDLLIHDVETNPGP

SEQ ID NO: 311 - protein, 30 aa, Artificial Sequence
  Synthetic
VFGLYRIFNAHYAGYFADLLIHDIETNPGP

SEQ ID NO: 312 - protein, 30 aa, Artificial Sequence
  Synthetic
VFGLYSIFNAHYAGYFADLLIHDIETNPGP

SEQ ID NO: 313 - protein, 30 aa, Artificial Sequence
  Synthetic
VLPCACGRATLDARRLLLPVGGGVERNAGP

SEQ ID NO: 314 - protein, 30 aa, Artificial Sequence
  Synthetic
VLPCTCGRATLDARRILLLISGDVERNPAP

SEQ ID NO: 315 - protein, 30 aa, Artificial Sequence
  Synthetic
VLPRPLTRAERDVARDLLLIAGDIESNPGP

SEQ ID NO: 316 - protein, 30 aa, Artificial Sequence
  Synthetic
VLPRSLTREEREVARLLLKISGDVESNPGP

SEQ ID NO: 317 - protein, 30 aa, Artificial Sequence
  Synthetic
VMTTMMLQGPGASNFSLLKQAGDVEENPGP

SEQ ID NO: 318 - protein, 30 aa, Artificial Sequence
  Synthetic
VMTTMMLQGPGATNFSLLKQAGDVEENPGP

SEQ ID NO: 319 - protein, 30 aa, Artificial Sequence
  Synthetic
VTTDDFVVFTFRSAHQDVTLGGDVETNPGP

SEQ ID NO: 320 - protein, 30 aa, Artificial Sequence
  Synthetic
WDPTYIEISDCMLPPPDLTSCGDVESNPGP

SEQ ID NO: 321 - protein, 30 aa, Artificial Sequence
  Synthetic
YFACTCERAALDAPRLPVLISGDVERNPGP

SEQ ID NO: 322 - protein, 30 aa, Artificial Sequence
  Synthetic
YFKIYHDKDMDYAGGKFLNQCGDVETNPGP

SEQ ID NO: 323 - protein, 30 aa, Artificial Sequence
  Synthetic
YFKIYHDKDMKYAGGKFLNQCGDVETNPGP

SEQ ID NO: 324 - protein, 30 aa, Artificial Sequence
  Synthetic
YFNIMHNDEMDYSGGKFLNQCGDVESNPGP

SEQ ID NO: 325 - protein, 30 aa, Artificial Sequence
  Synthetic
YFNIMHSDEMDFAGGKFLNQCGDVETNPGP

SEQ ID NO: 326 - protein, 20 aa, Artificial Sequence
  Synthetic
YHADYYKQRLIHDVEMNPGP

SEQ ID NO: 327 - protein, 30 aa, Artificial Sequence
  Synthetic
YKIKLVAPDKQLCNFDLLKLAGDVESNPGP

SEQ ID NO: 328 - protein, 30 aa, Artificial Sequence
  Synthetic
YKQKIIAPAKQLLNFDLLKLAGDVESNLGP

SEQ ID NO: 329 - protein, 30 aa, Artificial Sequence
  Synthetic
YKQKIIAPAKQLLNFDLLKLAGDVESNPGP

SEQ ID NO: 330 - protein, 30 aa, Artificial Sequence
  Synthetic
YKQKIIAPEKQLLNFDLLKLAGDVESNPGP

SEQ ID NO: 331 - protein, 30 aa, Artificial Sequence
  Synthetic
YKQKIVAPAKQLLNFDLLKLAGDVESNPGP

SEQ ID NO: 332 - protein, 30 aa, Artificial Sequence
  Synthetic
YKQKIVAPVKQTLNFDLLKLAGDVESNPGP

SEQ ID NO: 333 - protein, 30 aa, Artificial Sequence
  Synthetic
YKQPLIAPAKQLLNFDLLKLAGDVESNPGP

SEQ ID NO: 334 - protein, 30 aa, Artificial Sequence
  Synthetic
YKQQIIAPAKQLLNFDLLKLAGDVESNPGP

SEQ ID NO: 335 - protein, 30 aa, Artificial Sequence
  Synthetic
YKTAITKPAKQMCSFDLLKLAGDVESNPGP

SEQ ID NO: 336 - protein, 30 aa, Artificial Sequence
  Synthetic
YKTAITKPVKQLCNFDLLKLAGDVESNPGP

SEQ ID NO: 337 - protein, 30 aa, Artificial Sequence
  Synthetic
YKTALVKPAKQLCNFDLLKLAGDVESNPGP

SEQ ID NO: 338 - protein, 30 aa, Artificial Sequence
  Synthetic
YKTPLVKPDKQMCNFDLLKLAGDVESNPGP

SEQ ID NO: 339 - protein, 30 aa, Artificial Sequence
  Synthetic
YKTPLVKPEKQLCNFDLLKLAGDVESNPGP

SEQ ID NO: 340 - protein, 30 aa, Artificial Sequence
  Synthetic
YKTSIVRPAKQLCNFDLLKLAGDVESNPGP

SEQ ID NO: 341 - protein, 30 aa, Artificial Sequence
  Synthetic
YKTTLVKPAKQLSNFDLLKLAGDVESNPGP

SEQ ID NO: 342 - protein, 30 aa, Artificial Sequence
  Synthetic
YKVSLVAPEKQMANFALLKLAGDVESNPGP

SEQ ID NO: 343 - protein, 30 aa, Artificial Sequence
  Synthetic
YQTALTKPAKQLCNFDLLKLAGDVESNPGP

SEQ ID NO: 344 - protein, 30 aa, Artificial Sequence
  Synthetic
YQTALVRPAKQLCNFDLLMLAGDVESNPGP

SEQ ID NO: 345 - DNA, 110 bp, Artificial Sequence
  Synthetic
cccggcgtcttgaattcggaagcggagctactaacttcagcctgctgaagcaggctggagacgtggaggagaaccctggacctatggtgagcaagggcgaggagctgttc

SEQ ID NO: 346 - DNA, 48 bp, Artificial Sequence
  Synthetic
cccggcgtcttgaattcttagtacagctcgtccatgccgagagtgatc

SEQ ID NO: 347 - DNA, 29 bp, Artificial Sequence
  Synthetic
caccggtactgttggtaaagccaccatgg

SEQ ID NO: 348 - DNA, 36 bp, Artificial Sequence
  Synthetic
cccccccgaattcgacgttgatgcgagctgaagcac

SEQ ID NO: 349 - DNA, 40 bp, Artificial Sequence
  Synthetic
gccagcgccaggatcaacgtcccgggccgcgactctagag

SEQ ID NO: 350 - DNA, 40 bp, Artificial Sequence
  Synthetic
ctctagagtcgcggcccgggacgttgatcctggcgctggc

SEQ ID NO: 351 - DNA, 40 bp, Artificial Sequence; identical to SEQ ID NO: 349
  Synthetic

SEQ ID NO: 352 - DNA, 40 bp, Artificial Sequence; identical to SEQ ID NO: 350
  Synthetic

SEQ ID NO: 353 - DNA, 18 bp, Artificial Sequence
  Synthetic
gaattctagagtcggggc

SEQ ID NO: 354 - DNA, 18 bp, Artificial Sequence
  Synthetic
aggtccagggttctcctc

SEQ ID NO: 355 - DNA, 34 bp, Artificial Sequence
  Synthetic
gagaaccctggacctatggtcttcacactcgaag

SEQ ID NO: 356 - DNA, 33 bp, Artificial Sequence
  Synthetic
ccgactctagaattcttagacgttgatgcgagc

SEQ ID NO: 357 - DNA, 18 bp, Artificial Sequence
  Synthetic
atggaagacgccaaaaac

SEQ ID NO: 358 - DNA, 23 bp, Artificial Sequence
  Synthetic
tcgattttaccacatttgtagag

SEQ ID NO: 359 - DNA, 29 bp, Artificial Sequence
  Synthetic
atatcgaattctttgctgagtggggctag

SEQ ID NO: 360 - DNA, 29 bp, Artificial Sequence
  Synthetic
ctagtggatccccactgatggggagaatg

SEQ ID NO: 361 - DNA, 4 bp, Artificial Sequence
  Synthetic
atcc

SEQ ID NO: 362 - DNA, 7 bp, Artificial Sequence
  Synthetic
atgatag

SEQ ID NO: 363 - DNA, 20 bp, Artificial Sequence
  Synthetic
gaattctcacggctttccgc

SEQ ID NO: 364 - DNA, 20 bp, Artificial Sequence
  Synthetic
gatgcgagctgaagcacaag

SEQ ID NO: 365 - DNA, 19 bp, Artificial Sequence
  Synthetic
cccgccgccagctcaccat

SEQ ID NO: 366 - DNA, 21 bp, Artificial Sequence
  Synthetic
cgatggaggggaagacggccc

SEQ ID NO: 367 - DNA, 52 bp, Artificial Sequence
  Synthetic
gctctacagaacatgtctaagcatgctgtgccttgcctggacttgcctggcc

SEQ ID NO: 368 - DNA, 86 bp, Artificial Sequence
  Synthetic
gctctagcttggaaatgacattgctaatggtgacaaagcaacttttagcttggaaatgacattgctaatggtgacaaagcaacttt